(19) United States
(12) Patent Application Publication
(10) Pub. No.: US 2025/0074842 A1
(43) Pub. Date: Mar. 6, 2025

(54) SYSTEM AND METHOD FOR SINGLE REACTOR CARBON DIOXIDE CAPTURE AND CONVERSION TO HIGH PURITY METHANE WITH POTENTIAL ISOTOPIC ENRICHMENT

(71) Applicant: CIRCULARITY FUELS, INC., MENLO PARK, CA (US)

(72) Inventors: STEPHEN E. BEATON, STANFORD, CA (US); GAURAV KAMAT, STANFORD, CA (US); FORD McCLURE, BELMONT, CA (US); ANNA LEE TONKOVICH, GILBERT, AZ (US)

(21) Appl. No.: 18/822,253

(22) Filed: Sep. 1, 2024

Related U.S. Application Data
(60) Provisional application No. 63/536,039, filed on Aug. 31, 2023.

(51) Int. Cl. C07C 1/12; B01D 53/02; B01J 20/04; B01J 23/46; B01J 23/89

(52) U.S. Cl. CPC C07C 1/12; B01D 53/02; B01J 20/041; B01J 23/462; B01J 23/892; B01J 23/8946; B01D 2257/504; C07C 2523/46

(57) ABSTRACT

Captured carbon dioxide is converted into ultra-high purity hydrocarbons, particularly methane. A gas stream containing carbon dioxide is fed to a reactor containing both sorbent and catalyst, until the sorbent portions are substantially saturated with carbon dioxide; non-sorbed species are removed from the first reactor via vacuum and/or purge cycles with high-purity gas; hydrogen gas is introduced to the first reactor to a pressure above ambient; reactor temperature is raised to facilitate desorption of carbon dioxide; and the carbon dioxide is catalytically transformed with the hydrogen gas into methane by recirculating the gas through the reactor. Carbon dioxide adsorption and desorption occur within the sorbent portions, while methanation takes place on the catalytic portions assisted by the sorbent at a temperature substantially consistent with the desorption. Downstream upgrading steps may remove or reduce impurities to produce ultra-high purity methane for chemical vapor deposition or other processes.

### BACKGROUND

This application claims benefit of U.S. Provisional Application Ser. No. 63/536,039 filed Aug. 31, 2023, the entire disclosure of which is hereby incorporated by reference.

### FIELD OF THE INVENTION

The invention pertains to the field of generating high-purity and ultra-high-purity methane. Specifically, it involves the use of materials that both capture and convert carbon dioxide to produce hydrocarbons such as methane and ethane of sufficient purity for use in chemical vapor deposition (CVD) processes for diamond and graphene synthesis or other processes seeking high purity hydrocarbons for thermochemical processes or other applications.

### RELATED ART

Conventional processes to make high purity small hydrocarbons such as methane via atmospheric carbon capture for synthetic diamond growth are dedicated to multi-reactor processes—using one process to capture carbon dioxide and another to generate the methane. Vince et al (U.S. Pat. No. 9,994,970) highlights the multistep process required to first capture the CO₂, cool, and expand the gas, before separation and hydrogenation in separate process hardware. Shearman et al (U.S. Pat. No. 11,371,162) adds to the related art by specifying the filtration steps required to purify the CO₂ of any contaminants, and specifically remove nitrogen contamination from amine-based sorbents, before the methanation step. Recently, Shearman et al (U.S. Pat. No. 11,713,250) has also shown how an isotopic enrichment step between carbon capture and methanation can result in methane and a CVD product of methane with a specific isotopic signature.

In contrast to these high purity methane generation techniques, there is a growing body of methane generation technologies from a single material comprising two or more functions (DFM or dual functional materials): capturing the CO₂ and catalyzing the reaction of CO₂ with H₂ to CH₄. These processes, typified by Gonzalez-Velasco et. al. publication titled “Mechanism of the CO storage and in situ hydrogenation to CH₄ Temperature and adsorbent loading effects over Ru—CaO/Al₂O₃ and Ru—Na₂CO₃/Al₂O₃ catalysts” and Farrauto et al publication titled “Feasibility Study of Combining Direct Air Capture of CO₂ and Methanation at Isothermal Conditions with Dual Function Materials”, struggle with the fact that the same reaction chamber used for direct air capture (DAC) must then be used for methanation. Conventional teachings are that systems and reactors that regularly contact air are incapable of producing high-purity gas. The associated published works use a nitrogen purge gas for the primary purpose of preventing the safety challenge of mixing high concentrations of oxygen and hydrogen. From the standpoint of making gas for CVD application, this process utilizing a nitrogen purge introduces additional external contaminants into the product. Additionally, the catalyst in these designs is deposited only on the surface along with surface-deposited CO₂-capturing sorbent. This surface-deposited arrangement of catalyst and sorbent, as well as the traditional teaching that a significant portion of the adsorbed CO₂ is chemisorbed in a configuration that would be lost under vacuum, makes the use of low-pressure systems, especially vacuum systems, challenging and counterintuitive for producing high purity hydrocarbon gas.

These conventional systems typically delaminate their catalyst over time due to mechanical stress, in many cases expensive rare metals, through the use of low pressure. Finally, in Farrauto et al and said associated published works, there is no teaching to alter the process steps to selectively capture and convert carbon dioxide with ultrahigh purity or based on the isotope of carbon to produce an isotopically enriched methane that is up to ten-times or more valuable for CVD applications.

The resulting product gas in the Farrauto et al applications and publication is therefore highly contaminated with nitrogen and is of the same isotopic ratio as CO₂ that exists in nature. These gases are therefore of low value to applications requiring high purity methane. Although the nitrogen contamination can be removed via a nitrogen rejection unit based on cryogenic distillation or pressure swing adsorption to the level of purity for pipeline gas using existing technologies, this mixture would require energy-intensive, slow, and expensive purifications to reach a sufficient level of purity (<1 ppm N₂) for chemical vapor deposition applications. Moreover, the Farrauto et al process steps do not incorporate any downstream purification methods required for high purity methane due to both the limited applications and the batch nature of their process.

### SUMMARY

The present invention details a process for producing high-purity hydrocarbons (e.g., methane, ethane) using one or more main or “core” reactors, wherein each main reactor comprises both a sorbent and a catalyst. The initial production of hydrocarbons comprises multiple steps conducted in the same hardware: CO₂ capture, reactor discharge of non-sorbed species, and hydrogenation. In most embodiments, a catalyst activation step is also conducted, after said reactor discharge of non-sorbed species, to activate the hydrogenation catalyst. This activation step comprises raising temperature to the reduction temperature of the provided catalyst, typically also causing the hydrogenation step to begin. The application of innovative vacuum steps, purge cycles, recycle loops, and/or temperature swings during these steps, in conjunction in certain embodiments with multiple main/core reactors and downstream upgrading steps, results in the generation of hydrocarbons suitable for use in chemical vapor deposition processes, such as those used in diamond and graphene growth. Preferred embodiments are capable of capturing CO₂ from air to produce a feed stream to said chemical vapor deposition processes that contains hydrogen, methane and/or ethane, and preferably very low nitrogen in the range from about 0.0001 to 10 ppm, and more preferably less than 1 ppm nitrogen. Certain embodiments are capable of capturing CO₂ from air to produce a feed stream to said chemical vapor deposition processes that contains hydrogen, methane and/or ethane, and less than 1 ppb nitrogen. Various embodiments of the invention overcome these challenges to efficiently generate hydrocarbons like methane potentially with enhanced isotopic purity and nitrogen contaminants less than 1 ppm in the hydrocarbon product stream.

### BRIEF DESCRIPTION OF THE DRAWINGS

FIG. 1A through FIG. 1D: Schematic visual summaries of one embodiment comprising four core steps for high purity hydrocarbons (methane featured here) from a main reactor containing both sorbent and catalyst. The temperature in Step 1 (FIG. 1A) is optimized for maximum CO₂ capture and minimal system complexity and cost, which occurs near ambient temperature and pressure conditions. Step 2 (FIG. 1B) is optimized for discharge of non-sorbed species from the reactor, also called “reactor purification”, which starts at about ambient temperature but can continue as temperature is ramped towards step three. The temperature for Step 3 depends on the catalyst, with FIG. 1C showing an exemplary catalyst activation temperature range of 225° C. to 300° C. Pure ruthenium metal forms labile bonds with oxygen that are easily broken at low temperatures (approximately 250° C.) whereas nickel or iron co-deposited with ruthenium require marginally higher temperature to quickly reduce to the catalytically active reduced metal state in the presence of a gas that comprises hydrogen. The temperature for Step 4 is optimized for high selectivity towards the hydrogenation reaction, which for CO₂ occurs between about 180° C. and 400° C. for most catalysts and products, with FIG. 1D showing an exemplary CO₂ hydrogenation temperature range of 300° C. to 350° C.

FIG. 1E shows an alternative process for Step 2 of FIG. 1B above and for Process P, wherein, instead of solely a hydrogen purge being used to purify/sweep the reactor from non-sorbed species, vacuum is applied to the reactor interior to pull out said non-sorbed species, followed by subsequent cycles comprising hydrogen purges through the reactor and then applied vacuum. FIG. 1E will be understood to show an alternative way to purify other embodiments of DFM-containing reactors of non-sorbed species in Step 2, such as to replace/adapt FIGS. 4B, 6B, and 8B for processes P1, P2, and P3.

FIG. 2: A diagram of certain embodiments of the process flow through which high purity hydrocarbons (methane is one preferred embodiment) are created from the main reactor process steps of FIG. 1, each of the two main reactors 20A and B shown in FIG. 2 containing both sorbent and catalyst and in parallel so that the feed stream may be switched from the first of the main reactors to the second of the main reactors, for example, when the first reactor transitions from the capture step to the purification step such that the process operates in a substantially continuous manner. The dashed lines in FIG. 2 indicate optional process steps and related piping.

FIG. 2A and 2B: A table split into two portions, FIG. 2A listing the reference numbers, the names/descriptions of corresponding processes or fluid streams of FIG. 2, and exemplary approximate compositions/concentrations of said fluid streams when the compositions are not step-dependent (“Constant Conditions”), and FIG. 2B listing exemplary approximate compositions/concentrations of said fluid streams of FIG. 2A when the compositions are step-dependent.

FIG. 3: A representative graph of the concentration of a carbon-containing effluent gas coming from the main/core reactor of FIGS. 1 and 2 at any given time in a single-pass hydrogenation run. As higher temperatures tend to desorb CO₂, reducing the duration of Step 3 is crucial in many embodiments to maximizing the carbon dioxide utilization. For example, the duration of Step 3 in certain embodiments may be preferably held to a range between about 1 second and 1 hour, and more preferably to less than about 20 minutes. Additionally, heating elements that selectively heat the catalyst, substrate, and the porous support upon which the catalyst is deposited to ensure that the catalyst is at the required temperature for catalysis by the time that carbon dioxide is released from the sorbent helps ensure maximum utilization of the previously sorbed carbon dioxide. In embodiments where Step 3 is prolonged, potentially due to catalytic activation through reduction of surface sites or other processes at higher temperatures, the system could be designed to send the reactor effluent during activation (substantially entirely hydrogen and CO₂) back to the same reactor or flow to a separate hydrogenation reactor. Less preferably, the system could be designed without flow for a period of time to allow the CO₂ that has been desorbed at said higher temperatures during activation to remain exposed to active hydrogenation sites. In this embodiment, the separate hydrogenation reactor contains a catalyst for conversion of CO₂ with hydrogen to hydrocarbons and may optionally also contain a sorbent for a DFM that enhances the conversion rate of CO₂.

FIG. 4A through FIG. 4D: A schematic visual summary of the four-step process of the main reactor of the embodiment listed as Example 1, wherein FIG. 4A portrays the CO₂ step, FIG. 4B portrays the reactor purification step, FIG. 4C portrays the catalytic activation, and FIG. 4D portrays the hydrogenation step.

FIG. 5: A process flow diagram of Example 1.

FIG. 5A and 5B: A table split into two portions, FIG. 5A listing the reference numbers, the names/descriptions of corresponding processes or fluid streams of FIG. 5, and exemplary approximate compositions/concentrations of said fluid streams when the compositions are not step-dependent (“Constant Conditions”), and FIG. 5B listing exemplary approximate compositions/concentrations of said fluid streams of FIG. 5A when the compositions are step-dependent.

FIG. 6A through FIG. 6D: A schematic visual summary of the four-step process of the main reactor of the embodiment listed as Example 2, wherein FIG. 6A portrays the CO₂ step, FIG. 6B portrays the reactor purification step, FIG. 6C portrays the catalytic activation, and FIG. 6D portrays the hydrogenation step.

FIG. 7: Process flow diagram of Example 2.

FIG. 7A and FIG. 7B: A table listing the reference numbers and names/descriptions of corresponding processes or fluid streams of FIG. 7, and exemplary approximate compositions/concentrations of said fluid streams.

FIG. 8A through FIG. 8D: A schematic visual summary of the four-step process of the main reactor of the embodiment listed as Example 3, wherein FIG. 8A portrays the CO₂ step, FIG. 8B portrays the reactor purification step, FIG. 8C portrays the catalytic activation, and FIG. 8D portrays the hydrogenation step.

FIG. 9: Process flow diagram of Example 3.

FIG. 9A and 9B: A table split into two portions, FIG. 9A listing the reference numbers, the names/descriptions of corresponding processes or fluid streams of FIG. 9, and exemplary approximate compositions/concentrations of said fluid streams when the compositions are not step-dependent (“Constant Conditions”), and FIG. 9B listing exemplary approximate compositions/concentrations of said fluid streams of FIG. 9A when the compositions are step-dependent.

FIG. 10: Process flow diagram of Example 4.

FIG. 10A FIG. 10B: A table split into two portions, FIG. 10A listing the reference numbers, the names/descriptions of corresponding processes or fluid streams of FIG. 10, and exemplary approximate compositions/concentrations of said fluid streams when the compositions are not step-dependent (“Constant Conditions”), and FIG. 10B listing exemplary approximate compositions/concentrations of said fluid streams of FIG. 10A when the compositions are step-dependent.

FIG. 11: Process flow diagram of Example 5.

FIG. 11A and FIG. 11B: A table split into two portions, FIG. 11A listing the reference numbers, the names/descriptions of corresponding processes or fluid streams of FIG. 11, and exemplary approximate compositions/concentrations of said fluid streams when the compositions are not step-dependent (“Constant Conditions”), and FIG. 11B listing exemplary approximate compositions/concentrations of said fluid streams of FIG. 11A when the compositions are step-dependent.

FIG. 12: Process flow diagram of Example 6.

FIG. 12A and 12B: A table split into two portions, FIG. 12A listing the reference numbers, the names/descriptions of corresponding processes or fluid streams of FIG. 12, and exemplary approximate compositions/concentrations of said fluid streams when the compositions are not step-dependent (“Constant Conditions”), and FIG. 12B listing exemplary approximate compositions/concentrations of said fluid streams when the compositions are step-dependent.

FIG. 13: Process flow diagram of Example 7.

FIG. 13A and FIG. 13B: A table split into two portions, FIG. 13A listing the reference numbers, the names/descriptions of corresponding processes or fluid streams of FIG. 13, and exemplary approximate compositions/concentrations of said fluid streams when the compositions are not step-dependent (“Constant Conditions”), and FIG. 13B listing exemplary approximate compositions/concentrations of said fluid streams of FIG. 13A when the compositions are step-dependent.

FIG. 14: Data from an embodiment of the invention (explained in Example 7) displaying the near thermoneutral dynamics of methanation of a sorbent-enhanced catalytic converter. The results shown in this figure illustrate the surprising results from the combination of sorbent and catalyst resulting in methane-production with limited to no exotherm with a temperature change of less than 20° C.

FIG. 15: Table of potential reactions from a dual function material system illustrating the varying standard enthalpy of formation from example reactions occurring on the dual function materials, for example, certain of the reactions occurring in the processes of FIGS. 13, 13A and 13B, and 14.

FIG. 16: Design of flow-through reactor with electrical leads for the creation of dielectric barrier discharge within the channels.

FIG. 17: A side view of a flowthrough reactor having an internal volume measuring about 2 liters (2L) with integrated joule electric heating that includes connection points for the recycle loop, thermocouples, flow, and vacuum ports. This reactor serves as the core reactor for both Example 7 and Example 8.

FIG. 18: A perspective view of the reactor of FIG. 17.

FIG. 19: An end view of the reactor of FIG. 17.

FIG. 20: A longitudinal view of the reactor of FIG. 17 viewed along the line 20-20 in FIG. 19.

FIG. 21A: An enlarged, detail view of the area of the reactor circled in FIG. 20 and labeled 21A, showing a portion of the sealant between the inner surface of the reactor wall and the flow-through monolith with DFM that is contained in the reactor, this being an exemplary sealing mechanism to temporarily affix the flow-through monolith into the reactor/pressure vessel that is capable of withstanding the vacuum and pressure of the process to make high purity methane.

FIG. 21B: An enlarged, detail view of the area of the reactor circled in FIG. 20 and labeled 21B, showing a portion of the reactor wall and the flow-through monolith with DFM that is contained in the reactor, wherein the monolith is portrayed with schematic lines (dashed lines) that represent the open-ended flow channels through the monolithic substrate.

FIG. 22 An end view of the reactor of FIG. 17.

FIG. 23: A longitudinal view of the reactor of FIG. 17 viewed along the line 23-23 in FIG. 22.

FIG. 24: An enlarged, detail view of the area of the reactor circled in FIG. 23, showing one O-ring, between the inner surface of the reactor wall and the flow-through monolith with DFM that is contained in the reactor, wherein another O-ring may be seen at the opposite end of the monolith in FIG. 23 and the O-rings serve to seal the monolith to the reactor wall inner surface, this also being an exemplary sealing mechanism to temporarily affix the flow-through monolith into the reactor/pressure vessel that is capable of withstanding the vacuum and pressure of the process to make high purity methane.

FIG. 25: A perspective view of the reactor containing monolith comprising a coating of DFM, as described in Example 4.

FIG. 26: A side view of the reactor of FIG. 25.

FIG. 27: A schematic drawing of the mechanics of a fuel cell used to alter the ratio of H₂ and CH₂ within the product gas as described in Example 8.

FIG. 28A, 28B, and 28C A basic piping and instrumentation diagram (P&ID), divided into three sheets due to the size and detail of the diagram, depicting process equipment and flow scheme that may be applicable to many embodiments of this invention including the processes in FIGS. 12 and 12A and B, Example 6, and processes including a fuel cell operating as shown in FIG. 27 and described in Example 8. FIG. 28A shows mainly the feedstock system, FIG. 28B shows mainly the reactor system to which the feedstock(s) of FIG. 28A are fed for producing methane or other hydrocarbons, and FIG. 28C shows mainly the downstream mass spectrometer, and fuel cell with vacuum pump for fuel cell exhaust.

FIG. 29: A representative graph of the concentration of a carbon-containing effluent gas at any given time, for example, as may be obtained from many embodiments of the invention when operating with ambient air as the feedstock, for example, from the main/core reactor in processes P4, P5, P6 of FIGS. 10, 11, 12. In contrast to the graph presented in FIG. 3 that demonstrates the effluent of the core reactor during a single pass of hydrogen, this graph shows the ability for the core reactor to produce high purity methane using the innovative vacuum/purge cycles, heating, and recycle loops described herein.

FIGS. 30 and 31: Graphs of the performance from a fuel cell when fed a hydrogen and argon mixture (FIG. 30) and a hydrogen and methane mixture (FIG. 31), showing the removal of hydrogen from the mixtures. Thus, FIG. 31 illustrates the fuel cell acting as a methane enrichment device that selectively oxidizes hydrogen thereby upgrading the feed gas containing significant percentages of hydrogen to a gas containing less than 1% hydrogen such that the methane purity is greater than 95% as defined on a dry gas basis. The dry gas basis is the gas composition that would be present after the condensation and substantially full removal of water. This graph is the result of an embodiment similar to that described in Example 8.

### DETAILED DESCRIPTION OF CERTAIN EMBODIMENTS OF THE INVENTION

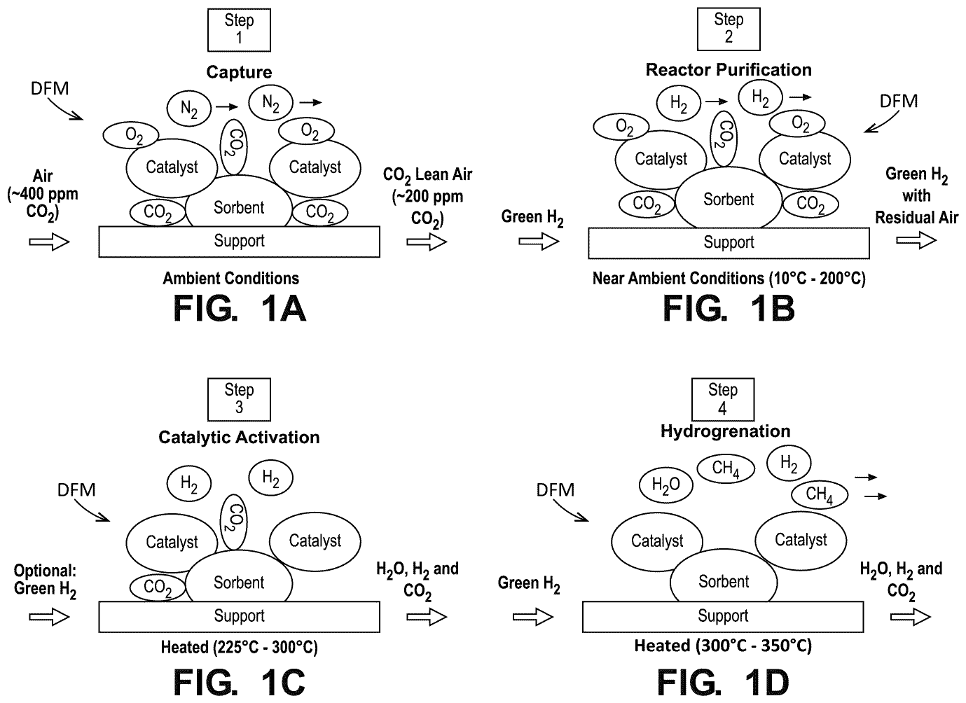

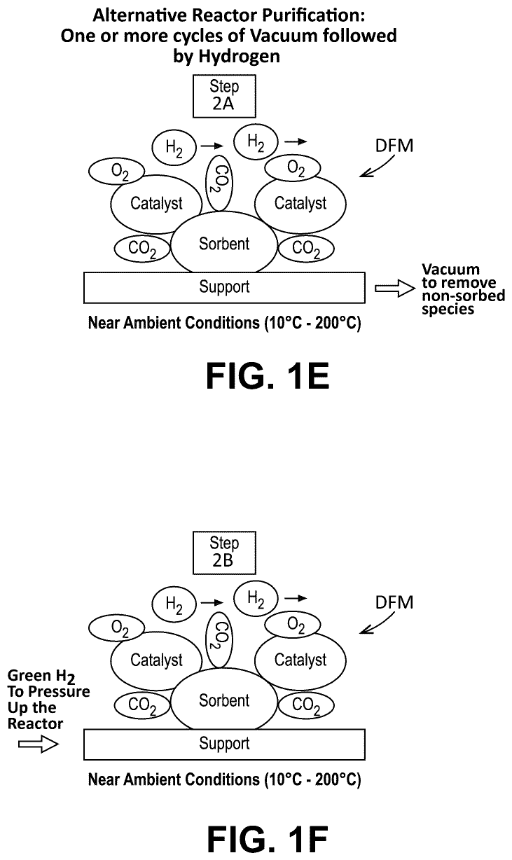

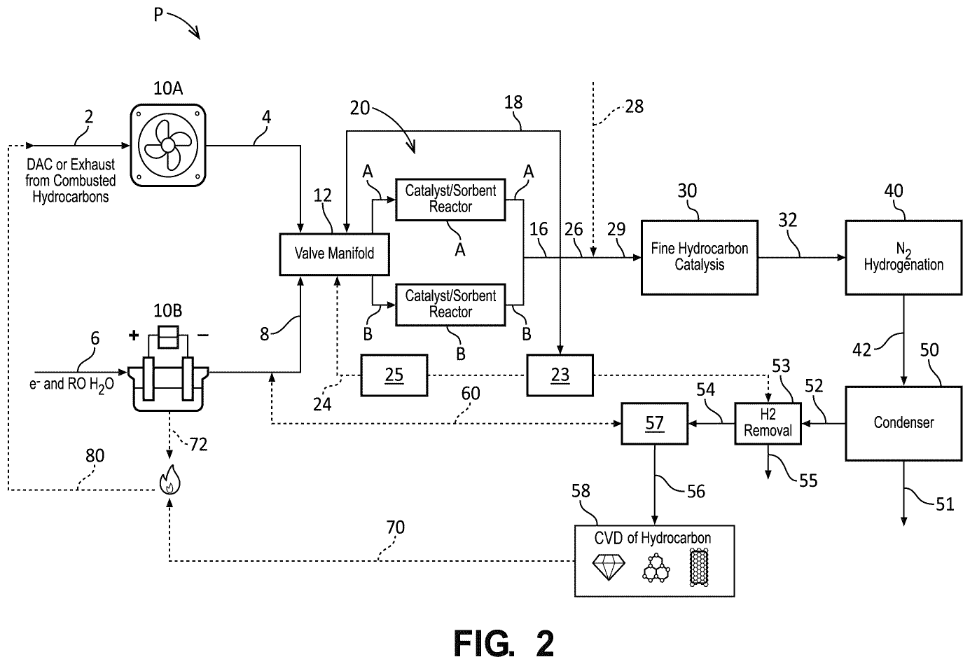

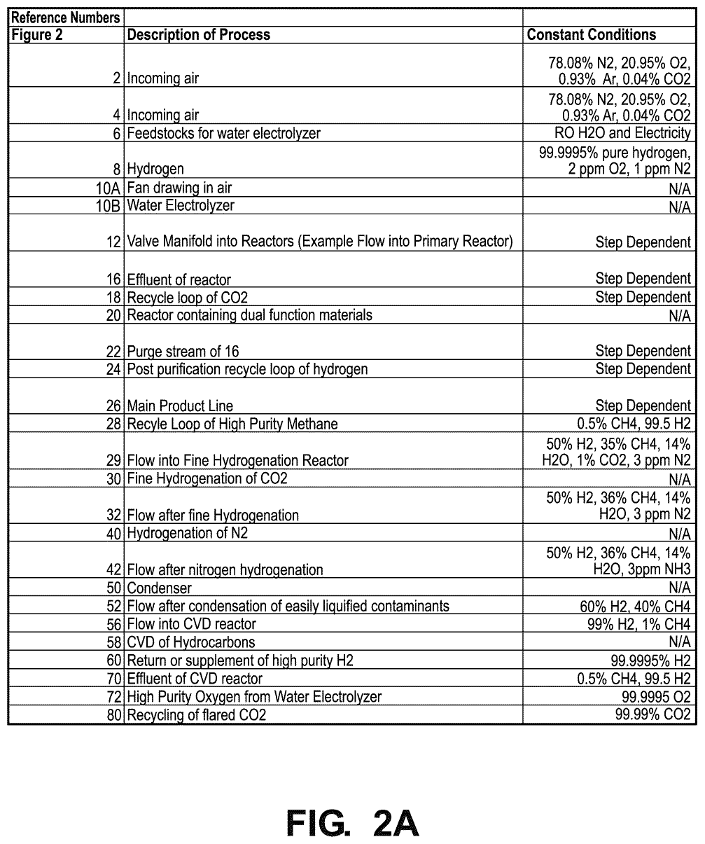

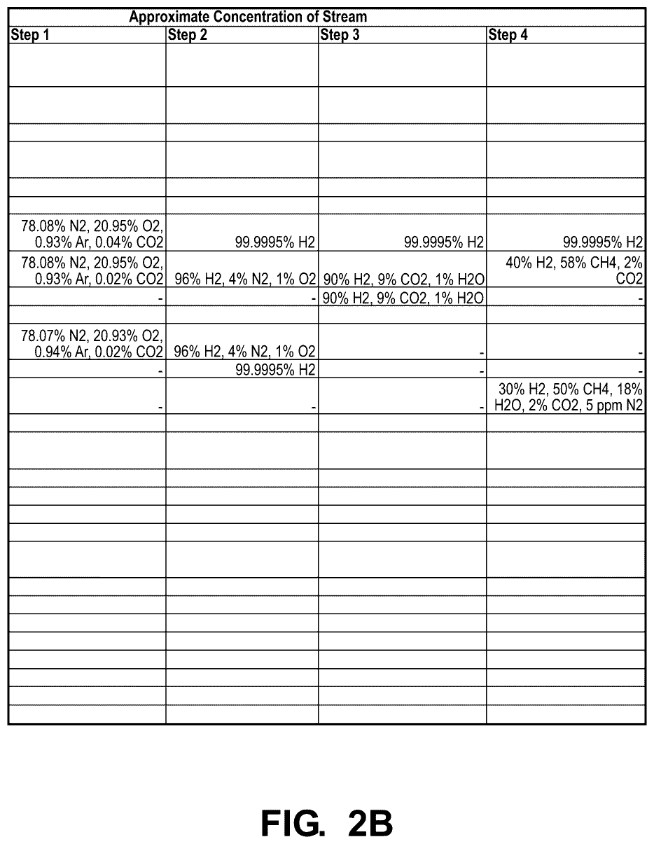

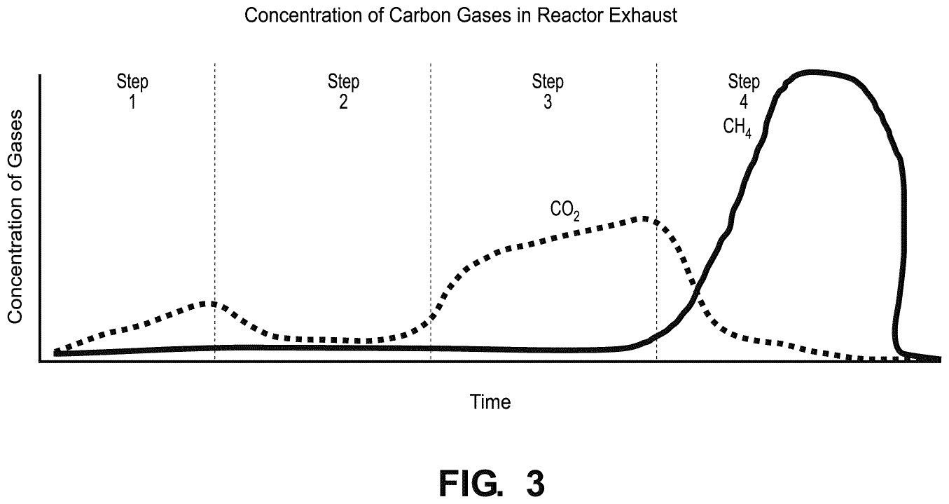

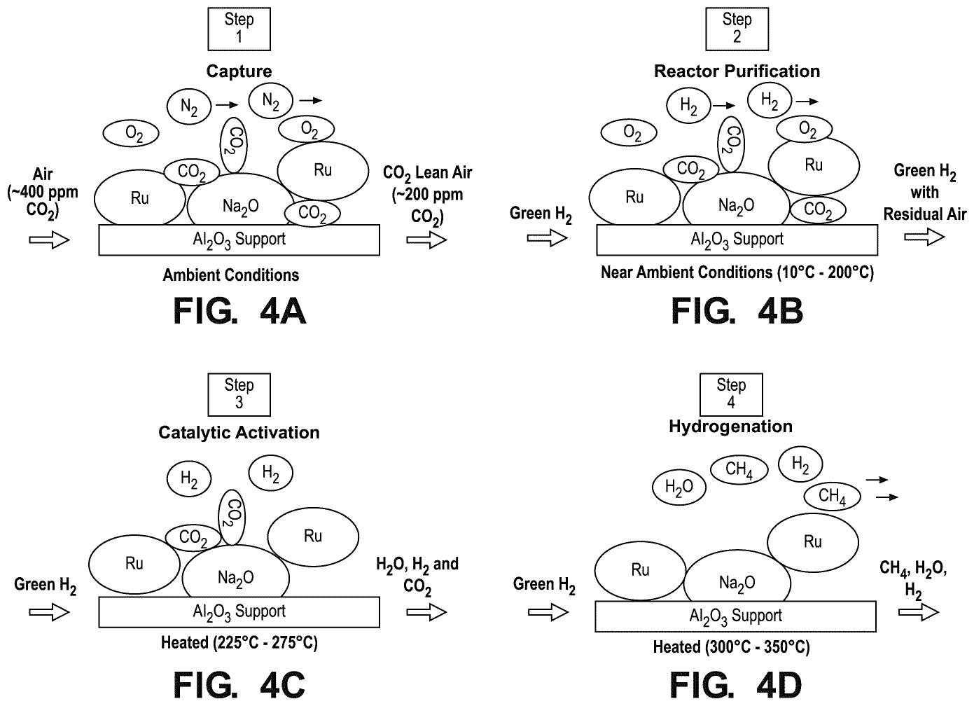

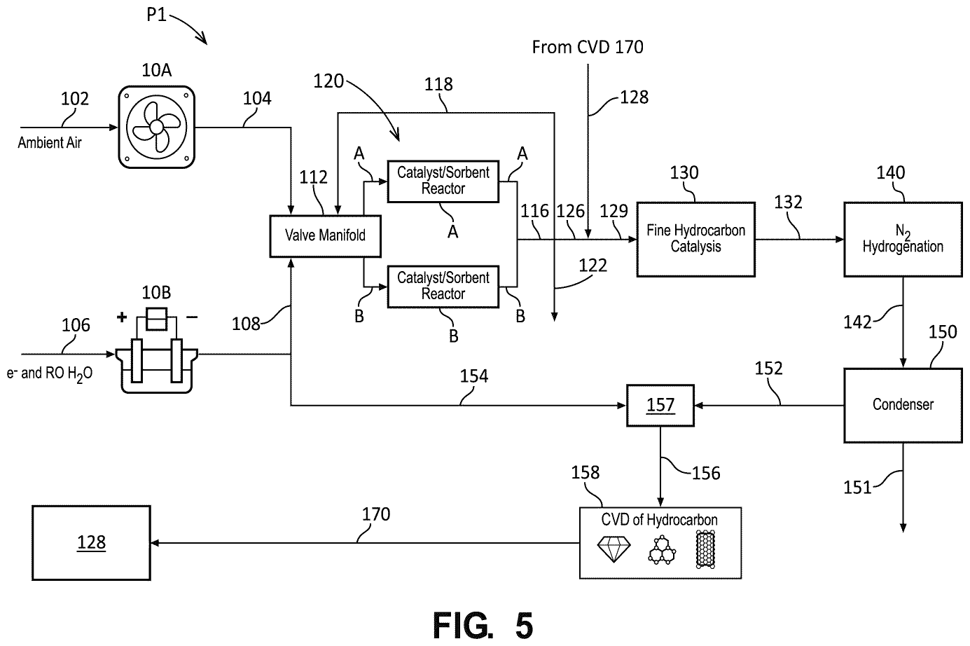

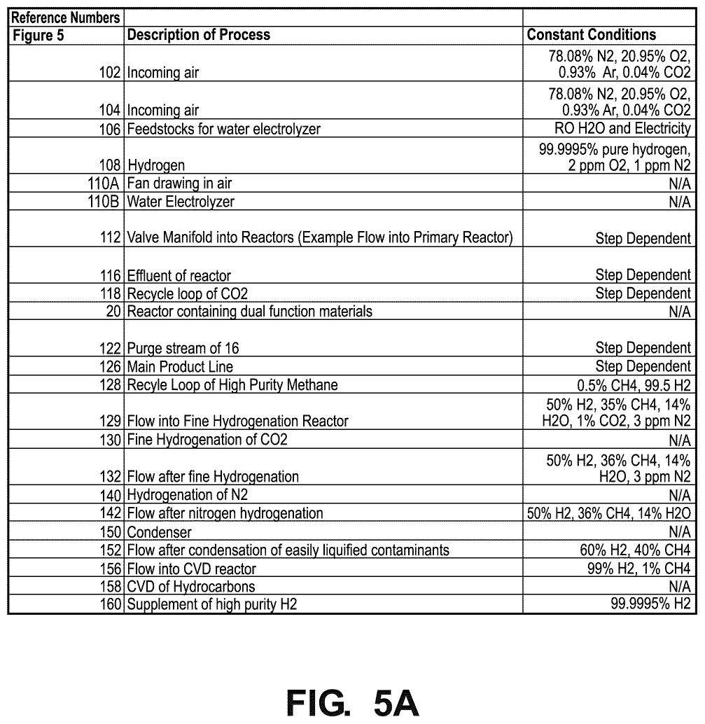

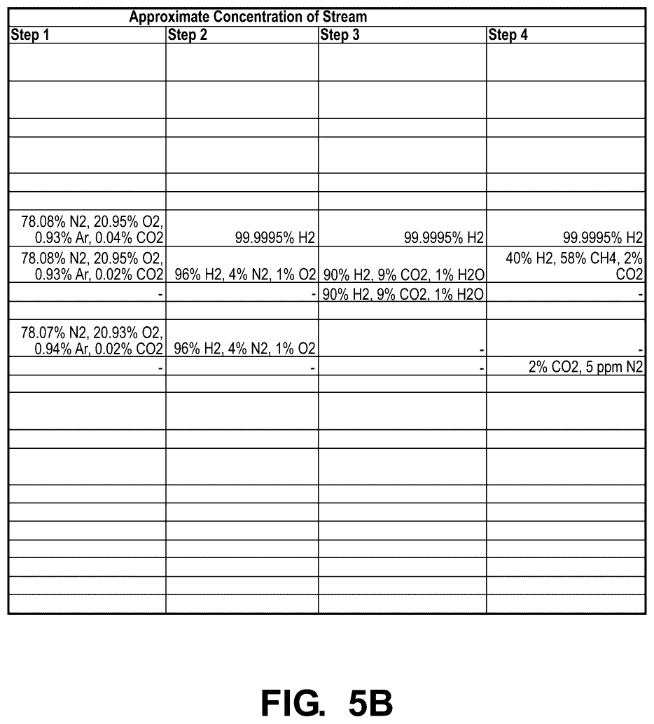

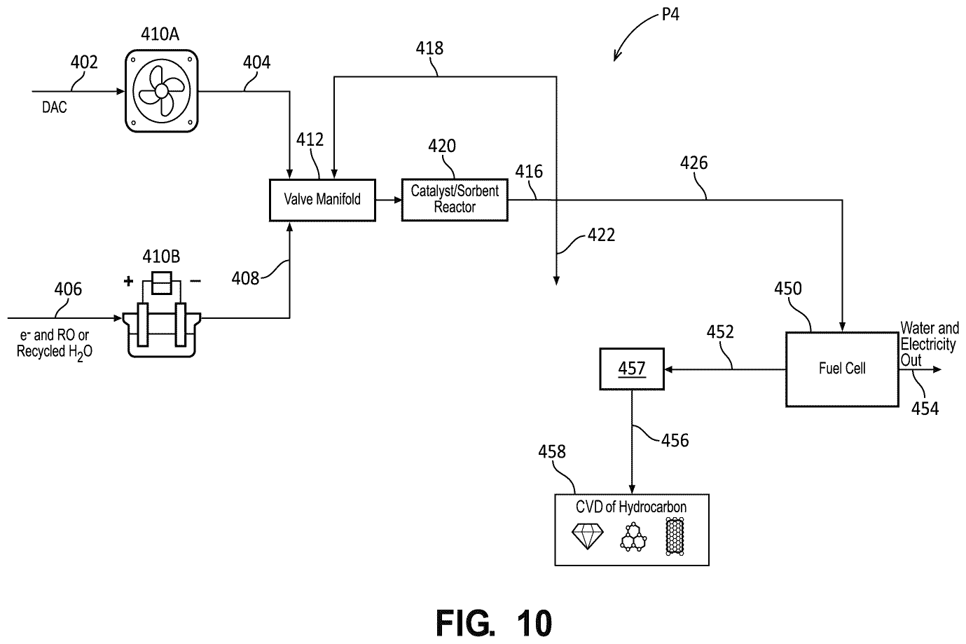

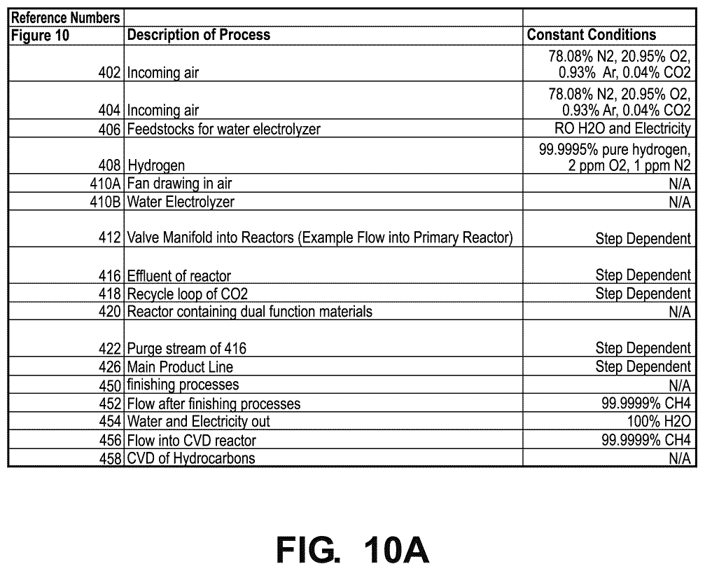

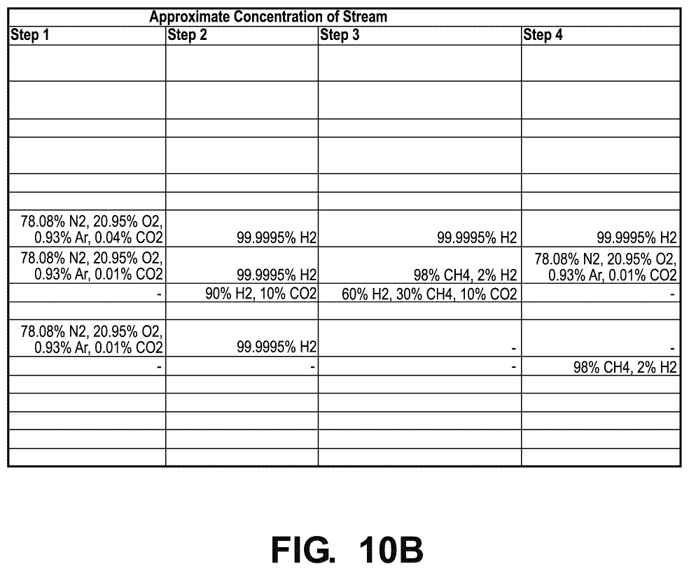

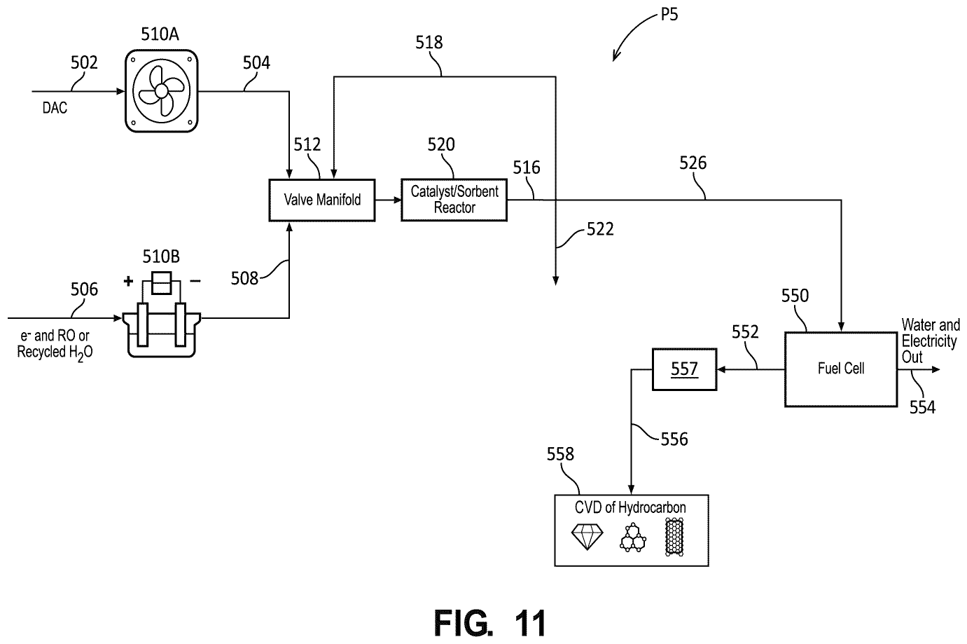

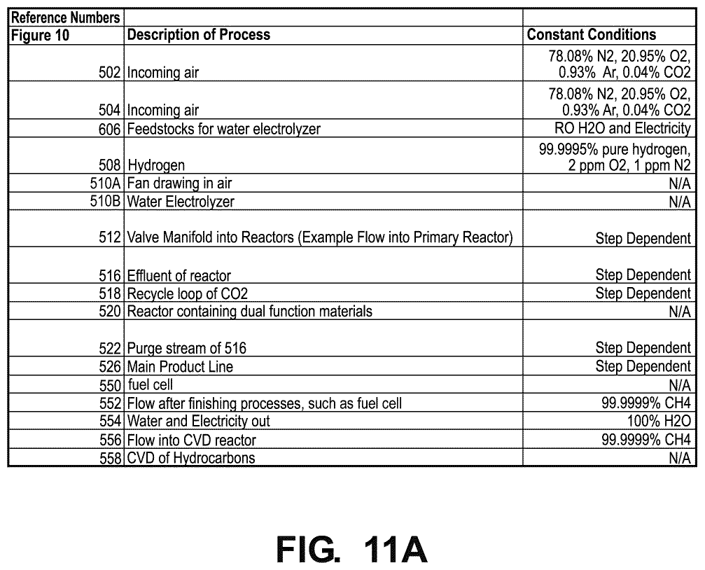

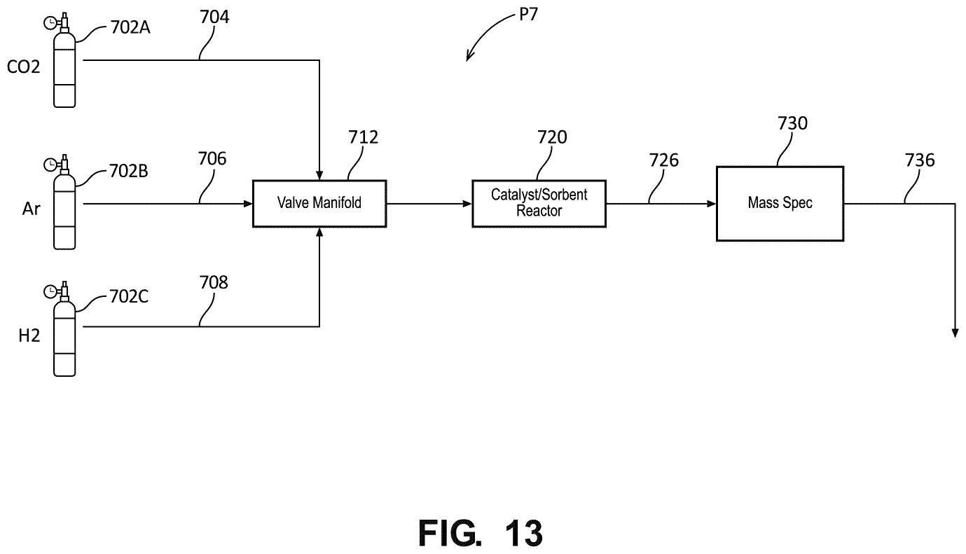

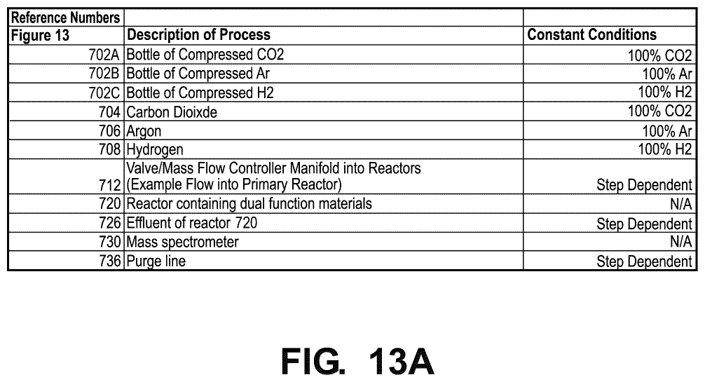

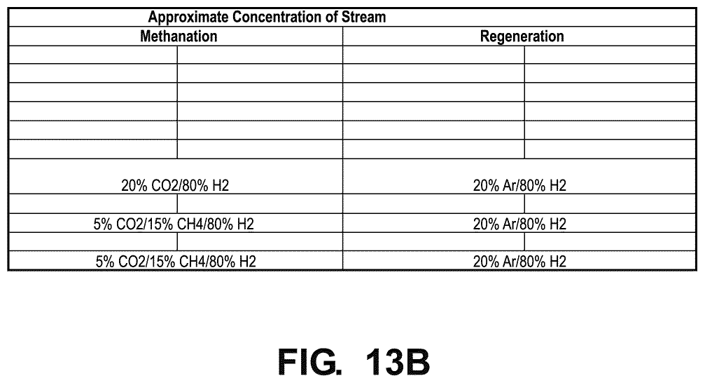

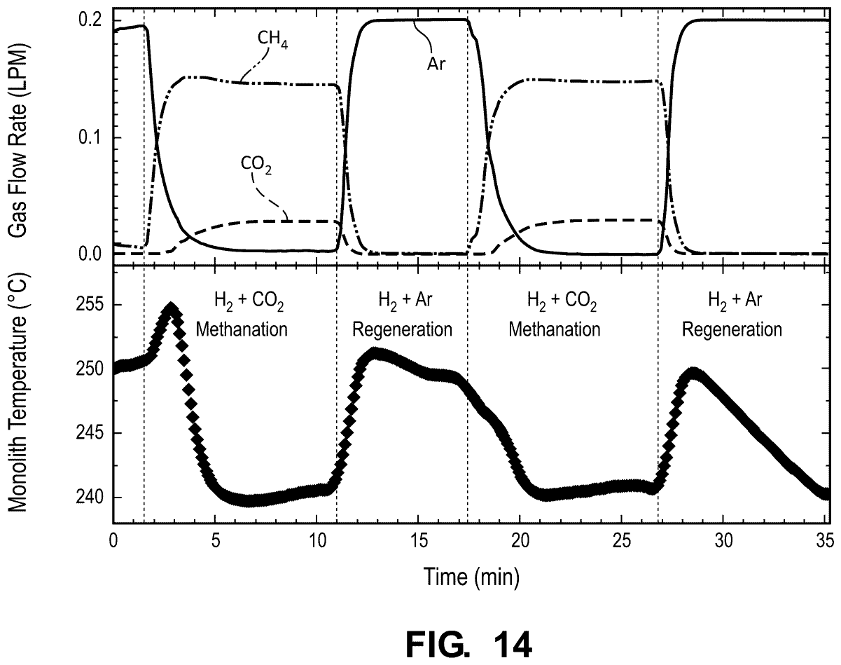

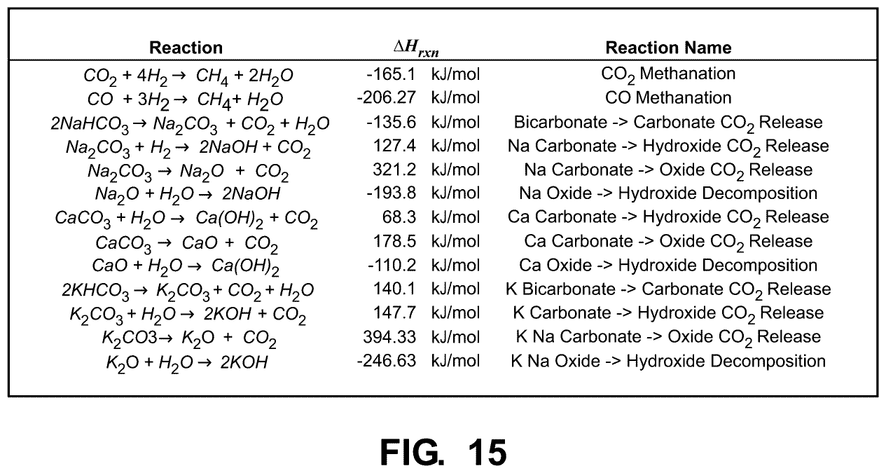

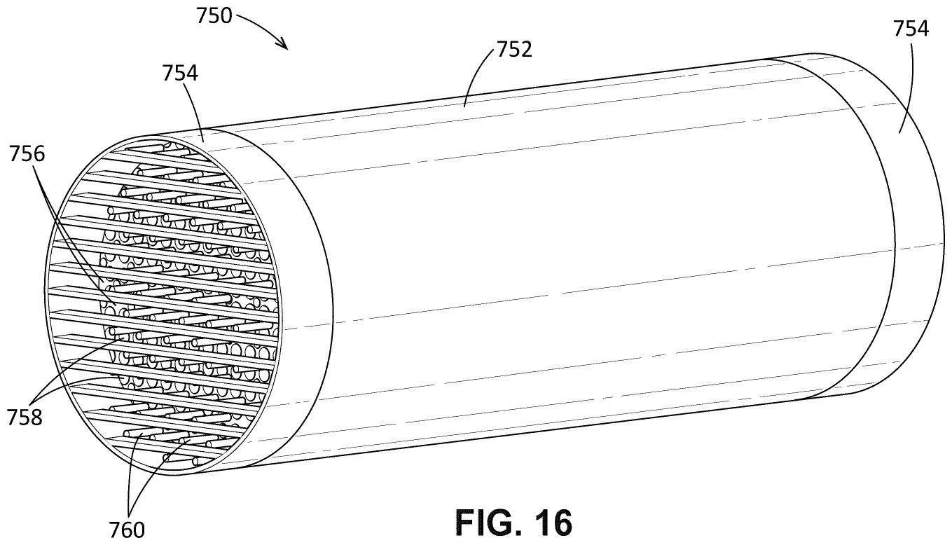

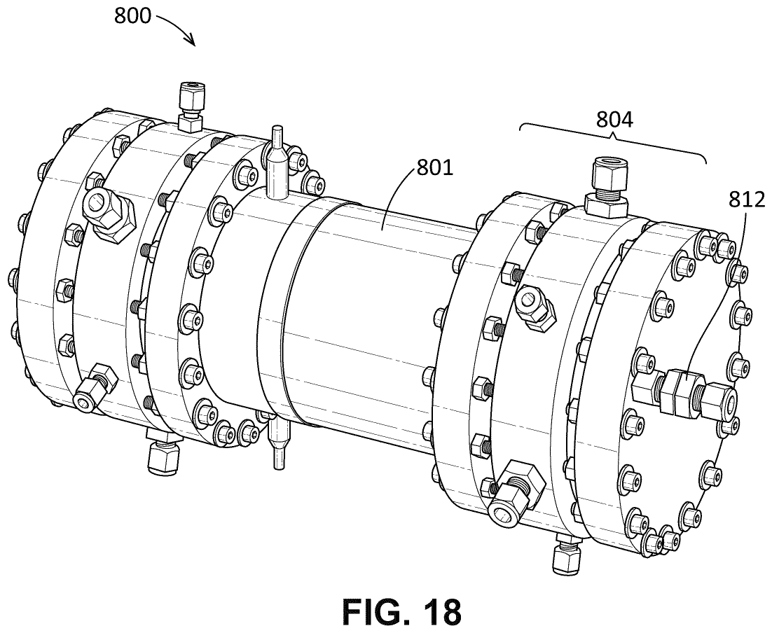

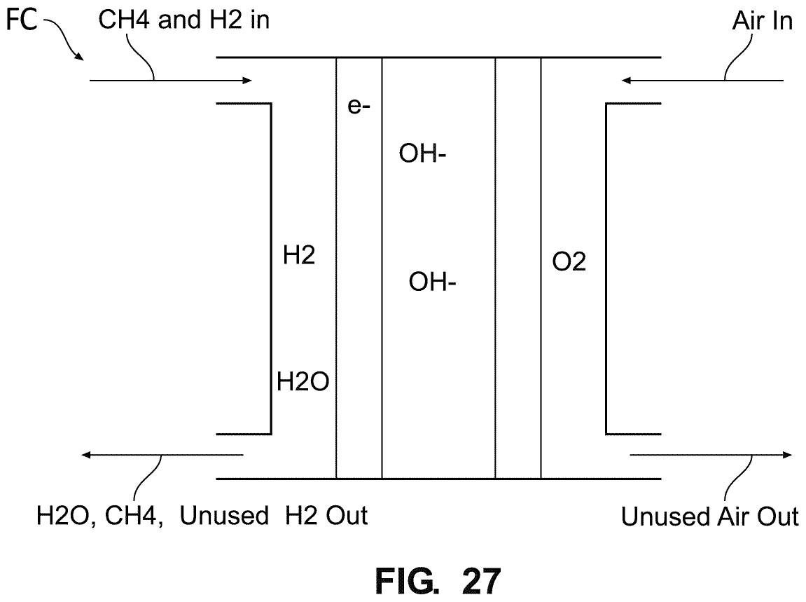

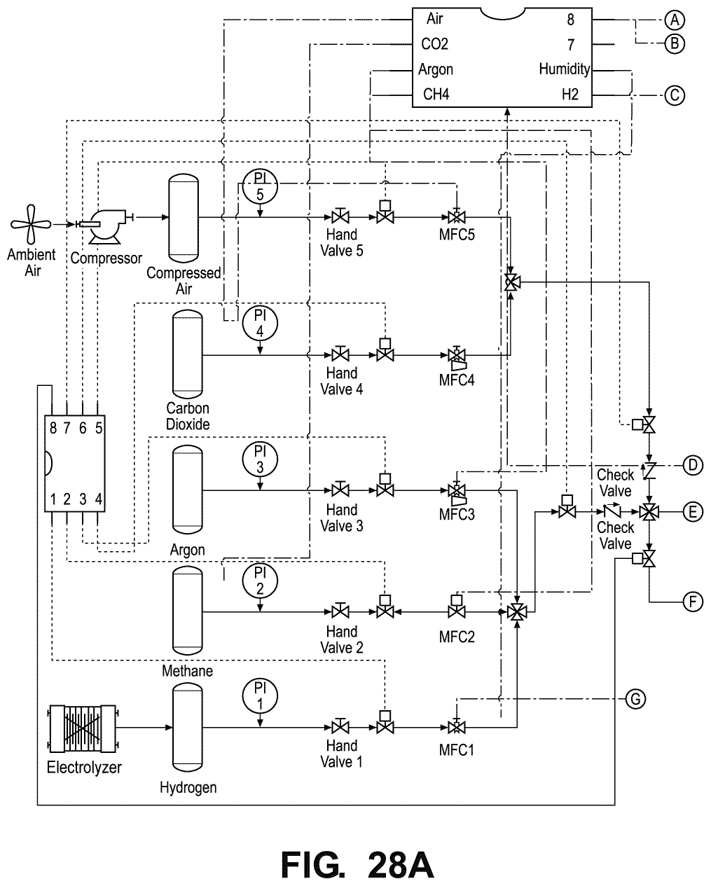

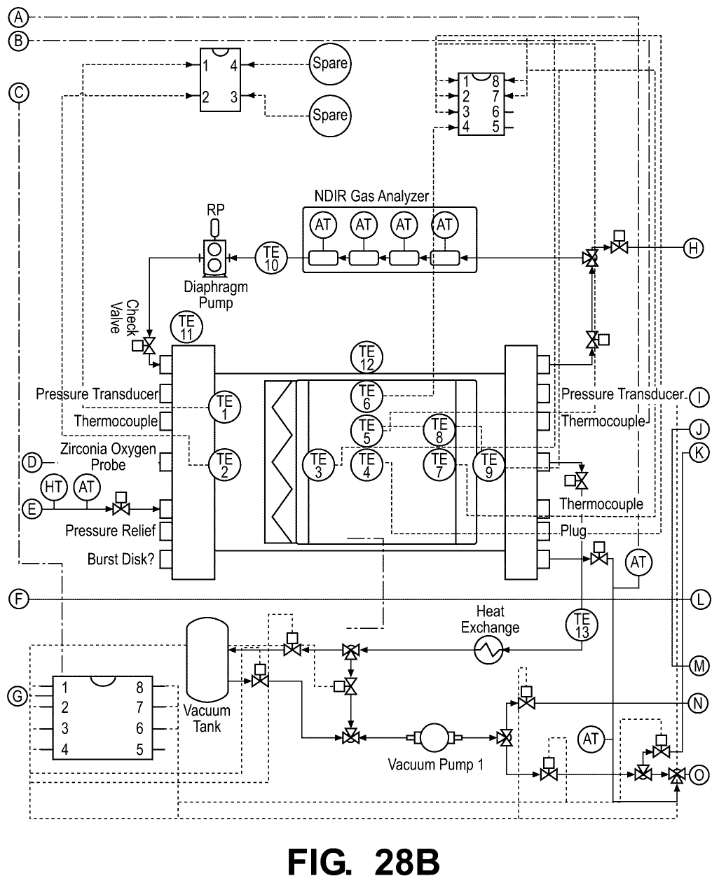

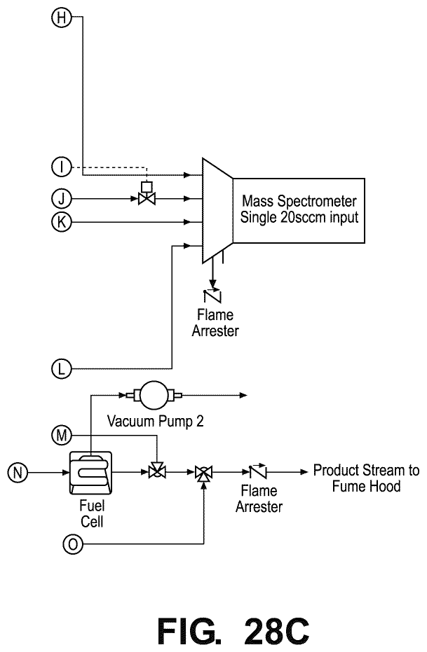

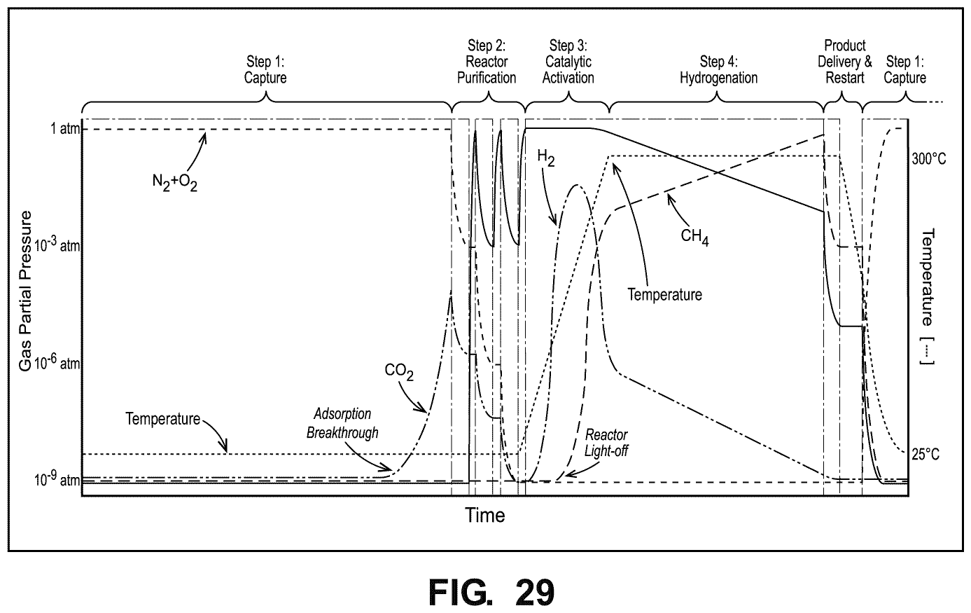

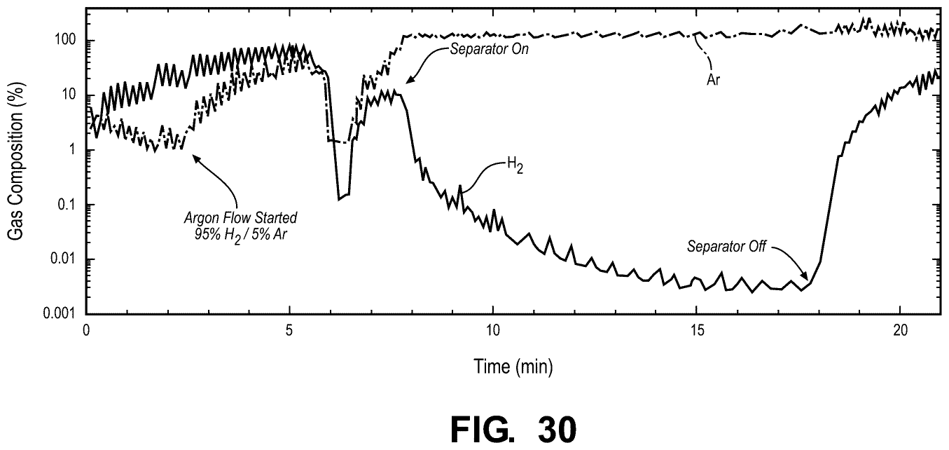

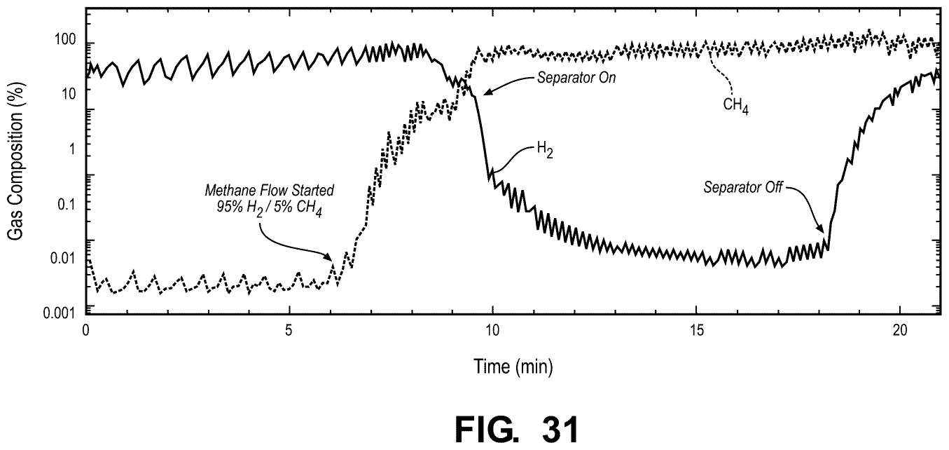

Referring to the Figures, there are shown several, but not the only, embodiments of the invented processes and apparatus. FIG. 1 summarizes one embodiment of the preferred four core process steps (Steps 1-4) for producing high purity methane in a single reactor 20 (also called the “main” or “core” reactor) containing both sorbent and catalyst. In the summary of the steps in FIG. 1, the coating (also called “washcoat”) is shown as consisting of a porous support (also called “carrier”), catalyst, and sorbent. Together these three layers/components, once deposited on a substrate are known as the “coating”; later percentages in this text and associated drawings will refer to the weight percent of these three layers/components as a percent of the entire coating consisting of support, catalyst, and sorbent. FIG. 2 shows a process P utilizing a reactor system 20, and it will be understood from FIG. 2 that the reactor system 20 comprises two of the main reactors (20 A and B) in parallel for sequential use and comprising multiple process options/alternatives for recycle streams and for upgrading of the hydrocarbon stream(s) downstream of reactor 20. FIG. 2A provides examples of compositions and concentrations for certain embodiments of the processes/streams of FIG. 2. FIG. 3 shows main reactor effluent (16) CO₂ and CH₄ concentrations over time, through the four steps of FIG. 1. FIGS. 4-9 show Steps 1-4 and process flow diagrams P1, P2, P3 of alternative embodiments described later in this document as Examples 1-3. FIGS. 5A, 7A, and 9A provide examples of compositions and concentrations for certain embodiments of the processes/streams of FIGS. 5, 7, and 9, respectively. FIGS. 10-13 show Steps 1-4 and process flow diagrams P4, P5, P6, P7 of alternative embodiments described later in this document as Examples 4-7. FIGS. 10A, 11A, 12A, and 13A provide examples of compositions and concentrations for certain embodiments of the processes/streams of FIGS. 10, 11, 12, and 13, respectively. FIG. 14 shows data, from the embodiment explained in Example 7 and FIGS. 13 and 13A, that illustrates the near thermoneutral dynamics of methanation of a sorbent-enhanced catalytic reactor. FIG. 15 shows potential reactions from a dual function material system such as may be provided inside the reactors of FIGS. 16-26, illustrating the varying standard enthalpy of formation from example reactions occurring on the dual function materials. FIG. 27 illustrates the operation of a fuel cell (also “separator” in the Figures) used to alter the ratio of H₂ and CH₄ within the product gas as described in Example 8. The fuel cell may be a commercially available product, such as POWERSELECT™ brand fuel cell. FIGS. 28A-C show a basic piping and instrumentation diagram (P&ID), which could be used for many embodiments of the invention. FIG. 29 shows the operating concept of the core reactor such as may occur in Example 6. FIGS. 30 and 31 are graphs of performance from a fuel cell or “separator” such as portrayed in FIG. 27, when fed a hydrogen and argon mixture (FIG. 30) and a hydrogen and methane mixture (FIG. 31), showing the removal of hydrogen from the mixtures. Thus, FIG. 31 illustrates the fuel cell acting as a methane enrichment device that selectively oxidizes hydrogen thereby upgrading the feed gas containing significant percentages of hydrogen to a gas containing less than 1% hydrogen such that the methane purity is greater than 95% as defined on a dry gas basis. FIGS. 30 and 31 result from an embodiment similar to that described in Example 8, and FIG. 31 especially illustrates beneficial performance such as may be achieved when a fuel cell is incorporated into the process.

The Examples may be briefly summarized as follows. Example 1 features/emphasizes a simple embodiment of the system with a recycle loop, the system sending effluent methane and hydrogen into the downstream purification steps as well as the use of temperature and flow-rate swings to isotopically enrich the methane used in the CVD process. Example 2 features/emphasizes changing the substrate, support, sorbent, and catalytic portions of the system; the pairing of ruthenium with base metal catalysts capable of performing the hydrogenation reaction under similar conditions; and the use of the hydrogen/methane CVD effluent in a hydrogen fuel cell to substantially recycle the inputs (electricity, water, and CO₂). Example 3 features/emphasizes using a catalyst that forms multi-carbon hydrocarbons, higher pressure and or temperature in Step 4 for hydrogenation, and combining all downstream purification steps into a single separation step. Example 4 highlights a full-scale commercial reactor operating at the current best practice for the reactor. Examples 5 and 6 highlight working bench-scale reactor operations producing high-purity methane from atmospheric carbon as detailed herein. Examples 7 and 8 highlight surprising performance of particular components of the system, the near-thermoneutral performance of sorbent-enhanced methanation catalysts and a fuel cell for selective hydrogen oxidation, respectively.

Certain embodiments of the present innovative process utilize a reactor system that combines the functions of carbon dioxide (CO₂) capture and its subsequent conversion with hydrogen into hydrocarbons such as methane (CH₄) within a singular operational unit where process steps are conducted at different times or as “displaced by time”. This integration is enabled by employing a unique matrix of dual-functional materials that serve both as sorbents for CO₂ capture and catalysts for its conversion.

The sorbent component of the reactor is engineered with high-performing carbon capture materials, including alkali metal oxides and alkaline earth metal oxides comprising potassium (K), lithium (Li), sodium (Na), calcium (Ca), magnesium (Mg), strontium (Sr), and/or barium (Ba), and combinations thereof. Alternatives could encompass amine functionalized materials, metal-organic frameworks (MOFs), zeolites, or combinations thereof. In an ideal embodiment, these materials have a high surface area, typically exceeding about 50 m₂/g, providing an enhanced adsorption capacity typically greater than about 500 μmol CO₂/g and from about 300 to 5000 μmol CO₂/g, thereby ensuring optimal capture efficiency of CO₂ directly from ambient air.

Simultaneously, the dual function material within the reactor houses a catalyst subunit, which may comprise active metals such as ruthenium, nickel, platinum, rhodium, copper, cobalt, or group VIII transition metals, their respective oxides, and combinations thereof. Alternatively, the catalyst could be a composite of group VIII transition metals in conjunction with group I alkali metals and group VII transition metals, promoting versatile and effective conversion of the captured CO₂ into useful hydrocarbons with carbon-carbon bonds. The catalyst can be applied onto the carrier material through various methods such as impregnation, ion-exchange, grafting, anchoring, post-colloidal deposition, spray coating, dip coating, or precipitation. Typically, the catalyst loading could range from about 0.25% to 30% by weight of the total coating and preferably from about 0.5 to 10 wt %, contingent upon the specific catalyst, the target reaction, and the process input conditions. The loading of the catalyst and sorbent have been combined to ensure that the reactions that occur during the desorption/methanation phase are substantially thermally balanced as defined by a gradient less than 50° C. and preferably a gradient less than about 20° C. FIG. 15 lists example reactions and their enthalpy of formation. Methanation is highly exothermic with a standard state heat of reaction at about −165 kJ/mol and about −178 kJ/mol at a reaction condition of 300° C. and desorption of CO₂ is highly endothermic (estimated with a heat of desorption from about 50 to 200 kJ/mol), thus balancing the two reactions is key to optimal energy efficiency and simplification of system requirements to eliminate the need for additional heat transfer equipment, fluids, pumps, and processing and/or design complexity. Modulating the rate of desorption is done by changing the loading of the sorbent and similarly modulating the rate of methanation is done by changing the loading of the methanation catalyst.

In an embodiment, both the sorbent and the catalyst are deposited onto a carrier material to create a cohesive operational unit that may be substantially fully or partially mixed throughout the layer or disposed as sequential layers wherein one or more layers of each material may be disposed upon each other. The carrier may comprise porous ceramic-based materials such as aluminum oxide (Al₂O₃), ceria (CeO₂), zirconia (ZrO₂), silica (SiO₂), or zeolites (SiO₂—Al₂O₃), and combinations thereof, each offering distinct advantages in terms of stability, thermal tolerance, and performance enhancement. In certain embodiments, the carrier, sorbent, and/or catalyst of the DFM, and the synthesis of the DFM, are specially adapted to ensure that the coated DFM is robust against vacuum pressure.

The carrier material is deposited on a multi-channel substrate such as honeycomb type monolith made from alumina, cordierite, or other ceramic. Alternatively, the carrier material is deposited on a multi-channel substrate formed by multiple parallel plates substantially similar in structure and style to ART™ Plate Reactors or similar microchannel or millistructured plate reactors. Alternatively, the carrier material is deposited on a corrugated metal ribbon that has been bent to form a flow-through substrate made up of a series of substantially parallel open flow channels. The metal ribbon may comprise stainless steel or iron-chromium-aluminum alloys (FeCrAlY) or other metals such as anodized aluminum. Alternatively, the carrier material is deposited onto a series of mesh filters also consisting of Nichrome or FeCrAlY. Alternatively, the carrier can be deposited onto a metallic based monolith type structure that may be manufactured using 3D printing or other manufacturing methods including brazing, welding, or diffusion bonding to create a regular or irregular open pore structure for flow through the device. In an alternate embodiment, the carrier may be disposed upon a metal or ceramic foam that has an irregular or regular first macropore structure for flow substantially through the reactor.

In summary, substrates for receiving carrier material and DFM are preferably rigid or substantially rigid structures that have multiple channels running longitudinally through the structure, each channel being open at both ends and not having any significant bends or blockages so as to reduce the pressure drop from the flow across the path length. One example of monoliths that may serve in supporting the carrier material and DFM are core-drilled ceramic (cordierite) monolith pieces where the channels are square and roughly 800 microns in height and width with the wall being approximately 100 microns in width. Another example is a metal monolith as described above that is spiraled, corrugated FeCrAlloy foil. The amplitude of the corrugation is also approximately 800 microns.

### Referring Specifically to the Figures

At least one main reactor 20, 120, 220, 320, 420, 520, 620, 720 containing one or more of the above dual-function materials, is provided in a process flow scheme P, P1, P2, P3, P4, P5, P6, P7, such as those shown in FIGS. 2, 5 (Example 1), 7 (Example 2), 9 (Example 3), 10 (Example 4), 11 (Example 5), 12 (Example 6), or 13 (Example 7), for example, and is operated to progress through the preferred four steps of CO₂ capture, substantial discharge of non-sorbed gas (also “reactor purification”), catalyst activation, and hydrogenation to produce hydrocarbons such as shown schematically in FIGS. 1, 4, 6, and 8.

### FIGS. 1-3

FIG. 1 shows an embodiment of the multiple cyclic steps that the dual function material (DFM) undergoes to accomplish CO₂ capture and hydrogenation to produce methane, in the process embodiment P of FIG. 2. The reference numbers shown on FIG. 2 are described later in this document, in the table of FIG. 2A, and/or as follows in this paragraph. CO₂-lean air flowing from the reactor as effluent 16 during CO₂ capture, or H₂ and residual air flowing from the reactor as effluent 16 during reactor purification, may be purged in stream 22. Valve manifold 23 is provided for receiving and then directing stream 22 to hydrogen removal 53 or to hydrogen purification 25 and then H₂ recycle via steam 24. Stream(s) 51 substantially remove(s) NH₃ and H₂O from the condenser system. Stream 55 sends power or ultra-high purity H₂ from H₂ removal system 53. CH₄ steam 54 from H₂ removal system 53 flows to valve manifold 57, which feeds ultra-high purity CH₄ to CVD 58. System 60 may supply high-purity H₂ from H₂ tanks or other H₂ gas lines for addition to the CVD feedstock via manifold 57 to stream 8, depending on industry preference. It should be noted that the dashed lines in FIG. 2 indicate optional process steps and related piping.

The initiation of the capture phase begins when the dual-function material reactor, embodying both sorbent and catalyst subunits, is exposed to an air or gas stream containing carbon dioxide (CO₂). The source of this air could originate directly from the ambient atmosphere and so CO₂ concentration may be about 350 to 500 ppm in dry air. Unless otherwise stated, all percentages of gas concentration stated in this text are volumetric percentages or ratios referring to the volumetric percentage or ratio of the gas in a dry gas composition. In other words, water vapor is neglected in citations of gas purity. Alternatively, the incoming stream could be partially or wholly supplemented by the emissions resulting from chemical vapor deposition process, hydrocarbon combustion, off-gas released from a fermentation process that comprises from about 1 to 99.999% CO₂ by volume such as anaerobic ethanol production fermentation or aerobic fermentation used in the production of specialty biological, chemical, protein, food, or energy products, anaerobic digester, landfill gas, and/or flaring processes. Before initiating the capture phase, the gas may undergo pre-processing steps to enrich the concentration of carbon oxides or a specific isotope of carbon oxides by cryogenic distillation, membrane separation, temperature swing adsorption, pressure swing adsorption, or selective oxidation.

The sorbent component of the reactor, engineered for CO₂ interaction, systematically adsorbs or absorbs CO₂ molecules from the air or other gas stream until a saturation point is attained fully or partially, this point representing a state of thermodynamic equilibrium when fully sorbed. In one embodiment of the invention, the capture phase operates under conditions of ambient temperature and pressure, thereby maximizing operational simplicity and cost-effectiveness. In another embodiment of the invention, the capture phase operates under higher pressure (at or about 900 up to 3000 torr) to minimize the size of the reactor and speed at which the saturation point is reached. In a third embodiment of the system, the flow rate, pressure, and temperature of the reactor are varied across the reactor to favor adsorption of CO₂ containing one specific isotope (e.g., carbon-12) over another (e.g., carbon-13).

The concentration of CO₂ in the exhaust stream from the DFM reactor is an essential parameter that informs the saturation status of the sorbent. In this innovative reactor system, CO₂ concentration is assessed via an integrated sensor mechanism, often employing advanced mass spectrometry, non-dispersive infrared (NDIR) sensors, or electrochemical sensors. Such sensors are typically placed strategically downstream of the reactor, where they monitor and record the concentration of CO₂ in the outgoing air or gas stream. Upon initiation of the capture phase, Step 1, the sorbent in the reactor effectively adsorbs or absorbs CO₂ from the incoming air or gas stream, resulting in a significantly reduced concentration (as defined by about 80% or greater reduction from the inlet composition when flowing air over the reactor at the ideal flow rate) or potentially near-zero levels of CO₂ in the exhaust stream as defined below about 80 ppm, below about 50 ppm, or from at or about 0 up to 30 ppm CO₂. As the sorbent approaches its saturation point, its capacity to adsorb additional CO₂ decreases. This change is reflected as a gradual or rapid increase in the CO₂ concentration in the exhaust air or gas stream, detected and reported by the CO₂ sensors. The hardware is preferably designed with open flow channels (also “open-ended flow channels”) then coated with a carrier and the DFM material for at least a portion of the length and at least a portion of the circumscribed channel walls. The open flow channels are preferably less than 2-mm in diameter, hydraulic diameter, or in width and/or height, to improve mass transfer of CO₂ from the bulk flow path to the DFM coated on the channel walls. An enlarged, detail view of the area of the reactor circled in FIG. 20 and labeled 21B, showing wherein the monolith is portrayed with schematic lines (dashed lines) showing the open-ended flow channels 831 through the monolith substrate. It will be understood from this disclosure that the many open channels extend longitudinally all along the length of the monolith and are located all or substantially all through the radial dimension of the monolith, and both ends of each channel 831 are open for accepting fluid into the channel at one end, allowing flow through the entire length of the channel, and out of the channel at the other end. Certain embodiments of the monolith substrate are known and may be obtained commercially, for example, vehicle catalytic converters or particulate filters from Corning™ (Celcor™ Technologies or DuraTrap™) or Applied Catalysts. For example, monolith substrates having open channels shaped to have square, round, corrugated radial or any other cross-section shapes. The inventors also envision alternative monolith substrates and/or alternative radial cross-section shapes. The average open flow channel hydraulic diameter preferably ranges from about 0.1-mm to about 2-mm, and many embodiments of the inventors' coating on such monoliths result in a DFM-coated monolith that surprisingly minimizes channel blockage during use in many of the process embodiments disclosed herein. For example, said DFM-coated monolith may accomplish CO₂ capture on the DFM walls while minimizing pressure drop during the first cycle of CO₂ capture, for example, minimizing the pressure drop across the DFM monolith to less than 1 bar per 50 mm of monolith substrate length, or preferably less than 0.01 bar per 50 mm of monolith substrate length, or more preferably between 0.002 bar and 0.001 bar per 50 mm of monolith substrate length when flowing at or about idealized flow rate for capture or conversion.

A critical indicator of essentially full sorbent saturation is the phenomenon known as ‘breakthrough’. This occurs when the concentration of CO₂ in the exhaust stream (16) begins to approach the concentration in the incoming air or gas stream, revealing that the sorbent is no longer effectively capturing CO₂. The breakthrough point, accurately detected by the system's integrated sensors, signals the need for the initiation of the reactor's purification or discharge of non-sorbed gas phase. See, for example, the CO₂ concentration curve at the transition from Step 1 to Step 2 in FIG. 3. See, also, the transition CO₂ concentration at the transition from Step 3 to Step in FIG. 4, wherein adsorbed CO₂ is hydrogenated to methane, which may be described as regenerating the sorbent for further cycles of operation.

As part of the seamless operation, in one embodiment of process P (FIG. 2), the transition from the capture phase to the purification phase for one reactor (20A) prompts the diversion of the incoming CO₂-containing air stream towards another reactor (20B) in the system. Thus, multiple, same or similar reactors (20A and B) are provided in parallel, rather than in series, for sequentially receiving feedstock (14), which is preferably air in Step 1 and then hydrogen in Steps 2-4, as shown in FIG. 1. While noted as green hydrogen in FIG. 1 and subsequent Figures, it is generally understood that hydrogen from any source may be used in the inventive process. While two reactors are shown, it will be understood that any number of these parallel, sequential reactors 20 may be provided. This simultaneous operation design, of multiple main reactors in different steps of the preferred process Steps 1-4, ensures the continuous and efficient capture and processing of CO₂, optimizing the overall performance of the innovative reactor system.

In certain embodiments, the reactor purification phase, Step 2, commences with the effective clearance of residual air from the reactor, by conducting a hydrogen purge until the non-sorbed species flow out of the reactor (FIG. 1B). In other embodiments, the reactor purification phase, Step 2, commences with the effective clearance of residual air from the reactor by one or more cycles of vacuum and hydrogen flow into/through the reactor (FIG. 1E and 1F). In one embodiment of the process, for example, a slight vacuum is applied within the reactor to facilitate this clearance. The application of vacuum pressure is controlled and is typically in the range of about 0.001 up to 250 torr, ensuring that the integrity and stability of the dual-function material system are maintained during this low-pressure step. In a preferred embodiment, the application of vacuum pressure is between at or about 0.01 torr and 10 torr. In a more preferred embodiment, the application of vacuum pressure is between at or about 0.1 torr and 1 torr. It is important to note that, in many embodiments, this vacuum step may be used in the place of the “hydrogen purge only” Step 2, for example, in place of the purge process shown in FIG. 4B, 6B, and 8B.

It is critical that this vacuum step is conducted within an optimized temperature range, typically between at or about 10° C. and 150° C. Careful temperature control during the vacuum step is necessary to minimize thermal degradation, delamination, and/or decomposition of the sorbent and catalyst materials, thereby preserving their functional capabilities for subsequent cycles of operation. Additionally, as some sorbents are hygroscopic and will have adsorbed water during the capture phase that will be released when the reactor is put under vacuum, the temperature and vacuum condition are controlled in tandem to optimize performance of the entire system. Finally, in some embodiments, these parameters will be tuned to promote the release of some CO₂ containing one carbon isotope (e.g., carbon-13) over another carbon isotope (e.g., carbon-12).

The evacuation time for this step has been calibrated, balancing the efficiency of the process and the stability of the materials. In conventional operation, the evacuation for a reactor with a volume of 1 m³ may take anywhere between at or about 3 seconds to 30 minutes, a duration based on the specific reactor design, the volume of the reactor, and the material system used. The duration of this vacuum according to preferred embodiments of the invention is between at or about 10 microseconds and 120 seconds, and in some embodiments 10 microseconds up to less than about 75 seconds, a duration optimized based on the specific reactor design, the volume of the reactor, the material system used, and the desired isotopic ratio. In some embodiments, the reactor is evacuated in two stages: first to rough vacuum with instrumentation such as a scroll pump, diaphragm pump, or rotary vane pump and later to high vacuum with instrumentation such as a turbomolecular pump, vapor jet pump, ion diffusion pump, or cryogenic pump.

Upon successful evacuation, the reactor volume is replenished with a flow of high purity hydrogen, ideally sourced from natural hydrogen wells or a sustainable method such as water electrolysis though can be delivered from any hydrogen source. This replenishment process effectively and substantially displaces remaining air in the reactor and primes the catalyst for the subsequent conversion process. The remaining content of oxygen and nitrogen in the reactor effluent after the hydrogen purge is less than at or about 1% by volume, preferably from about 0 to 0.1%, more preferably still from about 0 to 10 ppm, and most preferably less than at or about 1 ppm.

The cycle of vacuum application and hydrogen flow can be repeated as necessary, depending on the specific design of the reactor and the characteristics of the dual-function material system. In certain embodiments of the reactor, the cycle of vacuum application and hydrogen flow is done two to five times, or, in other embodiments, more than five times. The effluent of the reactor (16) is monitored for concentration of CO, CO₂, H₂, O₂, H₂O, and/or CH₄.

In certain embodiments of the reactor, the hydrogen flow is conducted under conditions of ambient temperature and pressure, thereby simplifying the process, and minimizing the energy requirements. In certain other embodiments, the hydrogen flow is conducted at a pressure between at or about 1000 and 25,000 torr to quickly clear the reactor of residual air. In a preferred embodiment, hydrogen flow pressurizes the reactor to between at or about 1000 and 10000 torr, and more preferably from at or about 3000 up to 10000 torr.

The reactor purification phase is integral to the overall functionality of the reactor system, ensuring the production of high purity hydrocarbons across multiple operational cycles with minor downstream purification steps. The surprising result that the effluent of the core reactor can reach high purity on commercially viable timescales, while maintaining the structural integrity of the reactor and its internal DFM coatings of catalyst and sorbent flouts conventional teachings that regularly introducing air into porous sorbent/catalyst materials are not suitable for high-purity processes.

The process steps comprising CO₂ capture at a first temperature, reactor evacuation with a vacuum step, and heating to at least one second temperature for a hydrogenation reaction to produce hydrocarbons with desorbing CO₂ is conducted in the same inventive hardware as displaced in time during the process cycle. It is advantageous to minimize process hardware rather than conduct the process using separate unit operations and hardware to capture CO₂ then desorb CO₂, to purify the CO₂, and then to react the purified CO₂ with hydrogen to produce methane or other hydrocarbons. Hardware is reduced with the inventive process along with associated piping between process steps. Further the inventive process utilizes energy produced from the highly exothermic Sabatier or methanation reaction to supply necessary energy to desorb CO₂ without the use of intervening complex heat transfer equipment as required with existing technology. The advantages of the inventive integrated process steps as displaced by time enable a more energy and exergy efficient compact process and system hardware for the capture and conversion of CO₂ to hydrocarbons.

When the effluent of the reactor has a significant hydrogen constituent defined by a concentration greater than about 1%, as it is in the purge cycles of Step 2, the flow can be diverted to a hydrogen fuel cell or other process instrumentation to convert, separate, or utilize hydrogen. Given that the reactor could be active in CO formation which typically poisons hydrogen fuel cell catalysts, this step is counterintuitive but could provide energy to different steps of the process. The anode is composed of platinum but may also be doped with additional metals such palladium, ruthenium, gold, cobalt, or other metals that enhance the resistance to potential carbon monoxide poisoning. This fuel cell can either be a standard fuel cell, chosen from many currently available fuel cell products, which converts hydrogen into energy or a fuel cell/electrolyzer that oxidizes hydrogen at the anode and produces hydrogen at the cathode (sometimes called an electrochemical hydrogen pump). This offers the benefits of providing power, purifying and pressurizing the hydrogen for reuse, and/or altering the concentration of the hydrogen gas in the stream. Additionally, the fuel cell could be designed to collect excess gas that is not oxidized by the fuel cell's anode such as CO₂ and CH₄. In such a design, the anode compartment may operate between a pressure of at or about 1 PSI and 150 PSI and the gas collected from the anode gas outlet may be recycled through the reactor or collected, pressurized, and stored as its own product stream. The power load on the fuel cell may be set up as a variable load to vary the driving force of hydrogen oxidation, which allows precise control over the hydrogen removed from the stream. Traditional teachings hold that fuel cells should operate under conditions where hydrogen purity is of the utmost importance, and purposefully introducing contaminants in the anode would render a fuel cell inoperable. By carefully calibrating the fuel cell components, power draw, flow rates, and pressures, the present inventors have shown surprising results of significantly enriching the methane in the exhaust of the anodic compartment of a fuel cell while maintaining fuel cell performance.

The process of catalytic activation, Step 3, commences with a controlled increase in the reactor's temperature under the flow or presence of high-purity (>99.999% purity) hydrogen. The primary objective of this step is to enact a reduction reaction that revitalizes or “regenerates” the catalyst by increasing the number of reduced metal sites, counteracting any oxidation that may have transpired during exposure to oxygen in the air or feed gas during the capture or sorption part of the process cycle. It is recognized that the catalyst may not be fully reduced nor is this essentially required, rather, the catalyst must be sufficiently reduced such that there are enough active sites to convert the sorbed CO₂ with hydrogen to methane or other hydrocarbons during the hydrogenation cycle.

Depending on DFM formulation and other process parameters, temperatures for catalytic activation and hydrogenation are expected to fall in a broad range, for example, for many but not necessarily all embodiments, a range of 50° C. to 400° C. for activation and 180° C. to 400° C. for hydrogenation. The catalytic activation step is conducted at temperatures that are elevated relative to the ambient temperature preferred for the CO₂ capture and reactor purification steps, for example, between 180° C. and 350° C., between 225° C. and 300° C. (FIG. 1C), or between 250° C. and 320° C. Such temperature parameters are optimized to facilitate the effective reduction of the catalyst, effectively reducing oxidation sites, thereby restoring the active catalyst state for the subsequent CO₂ conversion process. In certain embodiments and typically depending on what DFM is being used, there will be a “threshold” temperature, at which activation by reduction of the catalyst begins, that is higher than the reactor temperature during the CO₂ capture step (referred to as the “first temperature” earlier in this document, and higher than the preferred ambient temperature of the reactor purification cycle/step. The reactor is raised to the threshold temperature to reduce substantially all, or all, of the catalyst, and the activation cycle/step may be described as the entire “temperature-ramping period” in which temperature is raised between the reactor purification temperature and said “threshold” temperature at which reduction begins. Thus, the duration of the activation step will depend on the rate of said controlled increase described above. Upon reaching this “threshold” temperature, as noted above, most but possibly not all the catalyst may already be reduced/activated, and reduction may continue even after there are enough active sites to convert the sorbed CO₂ with hydrogen to methane or other hydrocarbons during the hydrogenation step/cycle, thus resulting in activation/reduction being ongoing during the period described herein as the hydrogenation step. In certain embodiments, the reactor temperature may be held at the threshold temperature for most or all of the hydrogenation cycle. In certain other embodiments, the reactor temperature may be raised further to a desired higher hydrogenation temperature; for example, see FIG. 29 wherein reactor temperature is ramped from the purification step temperature to about 300° C. for the hydrogenation step, but CH₄ partial pressure starts to increase (“reactor light-off” indicating beginning of hydrogenation) while the reactor temperature is only about midway between the purification temperature and 300° C. In a distinctive embodiment of the reactor system, this catalytic activation phase is conducted under specific hydrogen pressure parameters, typically ranging between at or about 700 torr and 25,000 torr. Operating within this pressure range ensures a sufficient reduction process for the catalyst, enhancing the overall operational efficiency of the reactor system. The temperature of the system and the pressure of the hydrogen are two parameters that are tuned in tandem to ensure sufficient reduction of the catalyst. During the reduction of the catalyst, the effluent (16) may be monitored for presence of H₂O, or the reactor may be equipped with UV-visible spectrometers that can confirm the presence of reduced metal complexes. Alternatively, through process optimization a target control window for reduction conditions will be determined for a specific design size and catalyst mass and composition such that the recipe will be followed during streamlined manufacturing. Importantly, during the reduction of the catalyst step, the reactor effluent/flow may be directed towards reactor B (via recycle stream 18 and valve manifold 12) so that desorbed CO₂ is captured. In other words, stream 18 may be used to recycle the reactor effluent during the shoulder of the activation/reduction reaction containing hydrogen, CO₂ and a low amount of water, in certain embodiments being directed to a separate reactor embodying substantially similar catalytic materials. This allows for the simultaneous activation of multiple reactors, enhancing the system's capacity to process larger volumes of CO₂. This catalytic activation phase constitutes a crucial step in the reactor's operational cycle, ensuring that the catalyst is in an optimally active state to effectively facilitate the conversion of captured CO₂ into a high purity hydrocarbon stream.

In certain alternative embodiments, the exhaust gas resulting from this activation step is uniquely managed by directing reactor effluent/flow containing CO₂, which tends to occur during activation and may also occur during hydrogenation, back to the original or sole reactor for further processing of the desorbed CO₂. See FIGS. 10, 11, 12 illustrating a single reactor 420, 520, 620 and recycle 418, 518, 618 back to the single reactor via the valve manifold 412, 512, 612. This way, reactor effluent can be channeled to a recycling loop for potential reuse in the system, promoting operational sustainability.

Therefore, in certain embodiments, the recycle loop may commence during the activation step (Step 3), and the recycle loop may continue into and throughout the hydrogenation step (Step 4). In certain embodiments, recycle through said recycle loop may be conducted all through the activation and/or the hydrogenation steps, or may be done intermittently in a “stop and go” pattern as desired, in other words “transiently”. In certain embodiments, said recycle is conducted transiently when the methane reactor effluent contains less than 1 percent CO₂ by volume, or started again to be continuously or substantially continuously when the CO₂ in said reactor effluent rises above 1 percent by volume in the effluent. While in operation, the recycle loop directs the flow from the outlet back to the inlet and is capable of directing gas at any temperature. Thus, the recycle loop is compatible with the temperature ramp of the activation step.

Redirecting the gas from the outlet via the recycle loop ensures full capture of CO₂ that may be desorbed during the activation step and increases the residence time of the product gas within the reactor to achieve complete conversion. As the products are recycled in Steps 3 and 4, the recycle loop may also be used to condense water out from the reactor by decreasing the temperature of the gas below the dew point. In that case, the reactor may gradually decrease in pressure as five moles of gas are converted to one mole of gas. Thus, the recycle loop or the reactor may have additional inlet ports where the reactor can be supplemented with additional hydrogen gas as the hydrogenation reaction continues. Alternatively, the recycle loop may be used to further heat the gas, increasing the speed of the heat ramp during the activation step, which would result in the produced water remaining in the gas state. In such a case, the recycle loop or reactor could have additional hydrogen inlet, but the decrease in pressure would be approximately half of what would occur if the water were condensed within the recycle loop.

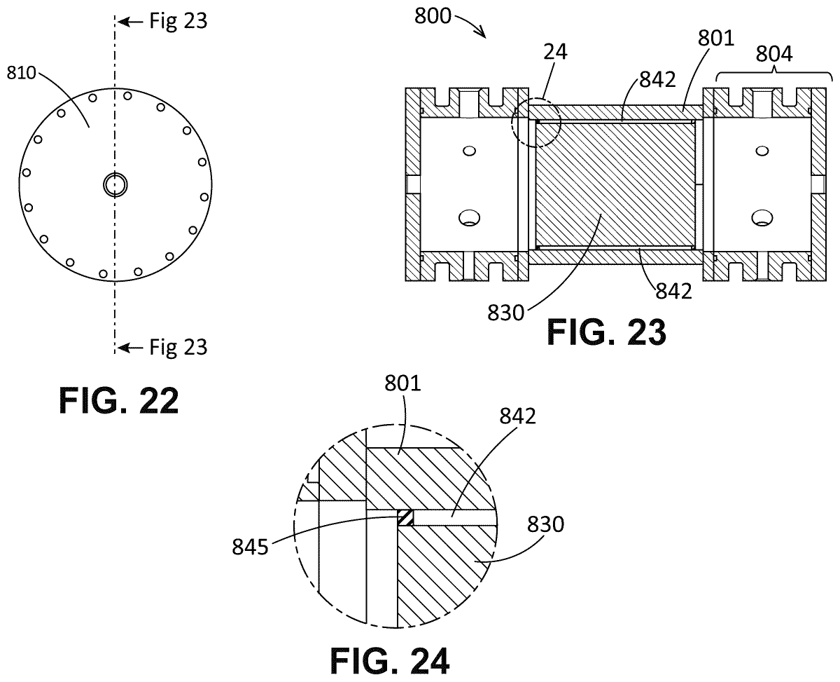

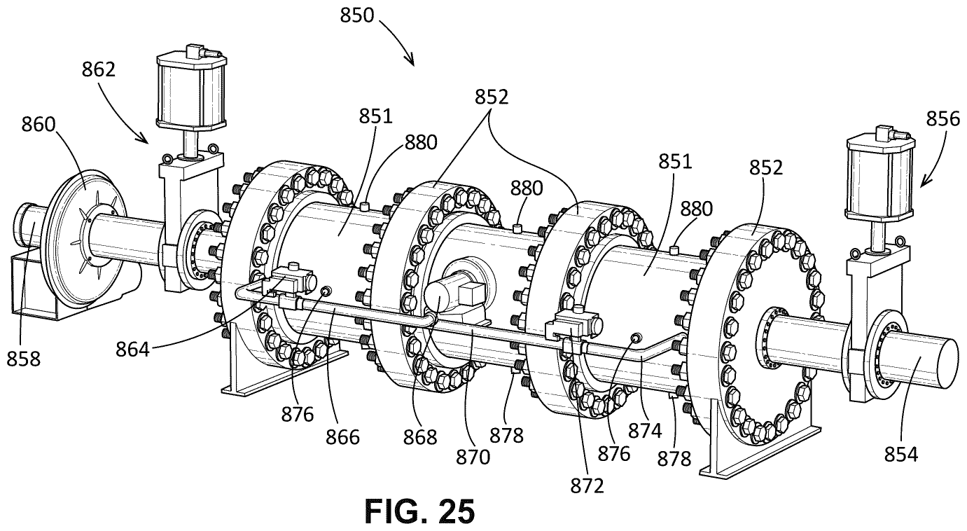

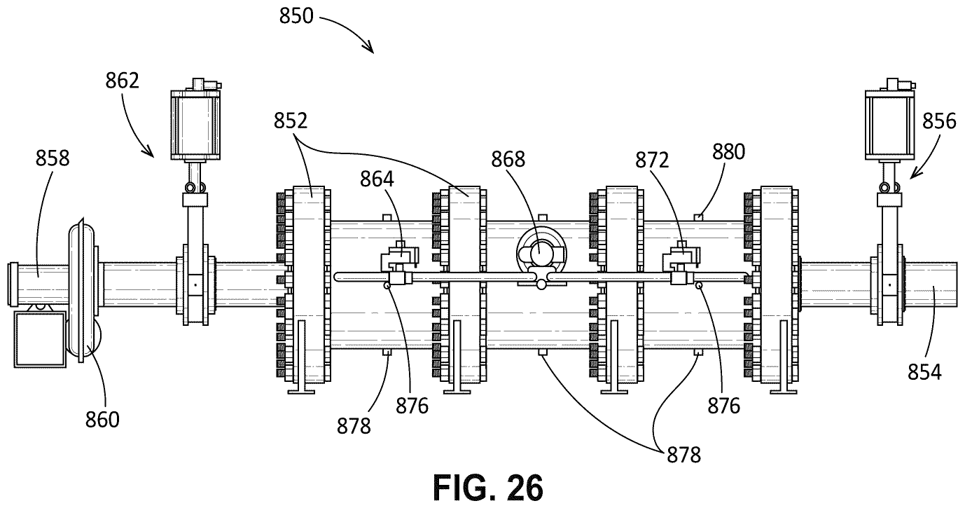

In one embodiment, a reactor with a 1000 L volume reactor interior space would have a pump recycling (also “recirculating”) the effluent during the activation step at a flow rate between about 5 SCFM and 1000 SCFM. In a preferred embodiment, the pump is recirculating the reactor gas at a flow rate between about 250 SCFM and 300 SCFM. Sec FIGS. 25 and 26. The recirculation loop may also contain additional volume in either a tank or additional gas lines that are pre-filled with hydrogen in order to supplement the amount of hydrogen available to recirculate through the reactor. The ability of the reactor to reach greater than about 99% CO₂ conversion to methane, as shown in FIG. 22, with a recycle loop is surprising. Conventional teachings hold that methanation catalysts are active for the water-gas shift reaction in the reverse direction generating CO that would build up in a recycle loop, poison the catalyst, and limit the activity and selectivity towards CH₄. Thus, most conventional methanation reactors are multiple reactors in series at different conditions, rather than a single reactor with a recycle loop. In other words, the prior art teaches against a recycle loop that feeds a DFM reactor's effluent back to that same reactor, because CH₄ would buildup and prevent full conversion due to thermodynamic limiting of the reaction, but the present inventors have found this not to be true. By contrast, the present inventors' carefully calibrated material and process overcome these limitations allowing the conversion of greater than about 99% CO₂ to CH₄.

Upon successful reduction of the catalyst, the reactor's temperature is adjusted to facilitate optimal hydrocarbon formation in Step 4. This temperature typically falls within a range of about 250° C. to 500° C., an interval that has been selected to maximize catalytic activity, selectivity, hydrocarbon yield, and/or any change to the isotopic ratio. By selectively converting CO₂ containing one isotope (e.g., carbon-12) over another (e.g., carbon-13), the final hydrocarbon product will be significantly isotopically enriched.

Hydrocarbon formation by hydrogenation step employs a calibrated pressure regime, typically within a spectrum of at or about 700 torr up to 25000 torr. Operating within this pressure range ensures an optimal environment for the catalyst to facilitate the conversion of captured CO₂ into the desired hydrocarbons, thus enhancing the reactor's operational efficiency. Most single carbon hydrocarbon products are generated at near atmospheric pressure of about 700 torr whereas longer hydrocarbons with higher counts of carbon-carbon bonds use progressively higher pressures through about 25000 torr. For example, methane is generated around atmospheric pressure (˜760 torr) whereas a system that favors ethane generation performs the hydrogenation step at higher pressures (from about 1000 to 5000 torr).

The exhaust gas from the reactor is monitored for the presence of hydrocarbons, employing advanced gas chromatography or mass spectrometry techniques, sensors, or other methods. This real-time monitoring aids in determining the progress and completion of the hydrocarbon formation process.

Once the monitoring system identifies that the exhaust gas stream (16) is comprised of hydrocarbons, the direction of the gas feed flow is strategically altered. For example, rather than being directed towards the recycle loop (18) (as previously described) for processing in a separate catalyst-containing reactor (B) or the purge stream (22), the hydrocarbon-rich gas stream is rerouted (26) to the primary product stream.

The hydrogenation cycle of Step 4 completes when the exhaust stream (16) displays only trace levels of hydrocarbons or is predominantly hydrogen signifying that the CO₂ sorbed in a previous stage is substantially depleted. At this stage, the comprehensive four-step process-encompassing capture, reactor purification, catalytic activation, and catalytic conversion-is restarted in the main reactor. However, depending on the intended inlet/feed stream, a vacuum step may be employed prior to the CO₂ capture step in some embodiments to evacuate flammable gas in the reactor without the use of an inert gas, thus maintaining a high purity environment. The dynamic monitoring of the hydrocarbon concentration in the exhaust stream, coupled with the strategic rerouting of gas flow, may ensure the continual production of hydrocarbons and the optimal utilization of the reactor system.

As described herein for the inventive process, different heating steps are used. The heat for these steps can be applied either through conventional means including resistive heating elements providing heat via convection, conduction, and radiation or less conventional mechanisms such as direct excitement and heating of the catalyst by stimulating plasma via microwave at 2450 MHz, dielectric barrier discharge (see FIG. 16), or induction heat addition methods. In an ideal embodiment, catalyst activation is performed through use of resistive heating elements such as Joule heaters as well as microwave power at 2450 MHz between 1500 and 2500 watts. Both resistive heating and microwave heat are applied until the catalyst reaches a higher temperature to catalyze the hydrogenation reaction at which point the heat from the hydrogenation reaction may be removed from the reactor using heat transfer fluids or alternatively and preferably, the thermal gradients are muted by the use of strongly endothermic CO₂ desorption coupled with strongly exothermic hydrogenation reaction such that the net instantaneous heat release is reduced and resulting excess heat generated by a net exothermic reaction can be more easily removed by the effluent gas stream and or reactor or system thermal losses.

The main product stream (26) derived from the innovative reactor system is processed through downstream upgrading steps to ensure the generation of ultra-high purity hydrocarbons. In certain embodiments, a crucial aspect of these upgrading steps is the incorporation of a secondary, catalytic process (30). This process serves to convert residual CO₂ into hydrocarbons, like CH₄. The concentration of CO₂ from the first reactor (26) may be between about 0.00001% and 20% by volume, but in preferred embodiments, the CO₂ concentration is expected to be in the approximate range of about 0.00001%-0.0005%, when the double reactor setup, recycle/recirculation loop, switching mechanism, and temperature are all optimized for maximizing the extent of methanation reaction in the dual function materials reactor (20). The rest of the composition of stream 26 is primarily comprised of hydrogen and methane, and the methanation reactor (30) reduces the percentage of CO₂ further in the effluent to less than about 0.00001% CO₂, but more preferably to between about 0.00001% and 0.0005% and most preferably less than about 0.00001% (32). Recycle stream (18) may be used to prevent/minimize buildup in the reactor (20) of CO₂ from the catalyst activation stage (Step 3) and reduce the CO₂ concentration from the first reactor effluent (26). For example, FIG. 3 shows that the CO₂ concentration increases during catalyst activation (Step 3) due to desorption caused by increased reactor temperature, but as the catalyst reaches the sufficient temperature to perform hydrogenation, CO₂ declines in Step 4 to the point wherein the reactor exhaust is substantially comprised of CH₄ with little or no CO₂ as defined by less than about 50 ppm. Use of a valve at the outlet of the reactor (20) and/or the manifold (12) can direct the recycle stream (18), from the first reactor (20A) during some or all of its Step 3, to a second reactor (20B) at its Step 1. Thus, the recycle loop (18) may be used to help control the composition of reactor effluent/exhaust (26) to allow downstream purification processes/equipment to be minimized or reduced in size.

The secondary catalytic step (30) may be conducted under different temperature and pressure conditions compared to the main reactor (20A or B), with parameters specifically optimized to maximize conversion efficiency. This reactor can utilize similar catalytic materials for performing the CO₂ hydrogenation reaction as the main reactor including nickel and/or ruthenium metal catalysts but may include additional catalyst materials such as cobalt, palladium, and rhodium or others that all have different temperatures for the methanation reaction. This step thus ensures the substantially complete utilization of CO₂ as defined by greater than 95% conversion and preferably from about 98 to substantially 100% conversion, further enhancing the economic viability and environmental impact of the reactor system. Thus, the combination of the main reactor system (20A or B) and the secondary catalytic process (30) may be described as providing multiple-stage, multiple-catalyst hydrogenation of CO₂.

A subsequent downstream step (40) targets the removal of trace nitrogen that may be present in the product stream, for example, by reacting N₂ with H₂ for subsequent NH₃ removal (50) or other trace nitrogen purification techniques. One embodiment utilizes the unique chemistry of the gas mixture, which contains excess hydrogen, to convert the trace nitrogen into ammonia (NH₃) or other nitrogen and hydrogen containing species. This strategic conversion not only aids in the purification of the hydrocarbon product stream but also may result in the production of ammonia (40), a valuable chemical with wide-ranging applications in sectors such as agriculture, cleaning, and industry.

In certain configurations, both the secondary catalytic conversion and the nitrogen removal steps could be performed within a single reactor. For example, iron and ruthenium catalysts have shown activity for the Haber-Bosch process and their presence in the secondary hydrogenation reaction could hydrogenate nitrogen contaminants in addition to CO₂. This integrated approach to downstream processing serves to streamline the overall operation of the system, reducing complexity and energy requirements, and promoting process efficiency.

These downstream upgrading steps (30, 40) play a pivotal role in the overall process, ensuring the production of ultra-high purity hydrocarbons, and thus, contributing significantly to the commercial value and sustainability impact of the innovative reactor system.

The final phase of the reactor system process preferably encompasses a purification procedure, employing condensation (50) as a key method for the removal of water and or other liquid contaminants, such as trace ammonia, which may be present within the reactor.

Given that high purity methane serves as the principal end product, often found diluted in a hydrogen medium, several strategic measures are taken to achieve the final working concentration. One approach involves the further dilution of the product with hydrogen to the required concentration level. For example, the product (52) will likely contain 5%-40% hydrogen and high purity hydrocarbons are used in a working concentration of 1% to 10% hydrocarbon in 90% to 99% hydrogen, making dilution desirable or necessary. Alternatively, the system can employ purification techniques to eliminate excess hydrogen. In one embodiment, the full product gas could be sent through a hydrogen-air fuel cell (see FIG. 27) or electrochemical hydrogen pump (53). This step would selectively oxidize the hydrogen content decreasing the hydrogen concentration of the product, and the remaining methane gas would be captured from the anode compartment exhaust of the fuel cell. In another embodiment, the product stream would undergo pressure vacuum swing adsorption, membrane separation, or temperature swing adsorption. However, in most embodiments, hydrogen will remain a significant constituent in the gas composition, typically accounting for 5% or more of the volume.

The versatility of the reactor and process design allows for co-location with chemical vapor deposition (CVD) facilities, enhancing operational synergy and efficiency. This integrated setup enables the direct flow of the product gas into the CVD chambers (58), significantly reducing the need for transportation and storage infrastructure.

In one particular embodiment, the hydrocarbons exhausted (70) from the CVD process (also, “excess” or “unused” hydrocarbons) could be treated in a combustive/oxidative process using substantially pure oxygen or steam (72), potentially sourced from the same water electrolyzer (10b) that produces the high purity hydrogen (8), to optionally recycle (80) from the combustion/oxidative process back to inlet stream 2. This procedure effectively combats any potential buildup of waste products within the system. It is generally understood that electrolyzer-produced oxygen will contain some amount of hydrogen that may be from about 0.001% to 2%.

In another embodiment, these hydrocarbons, typically with hydrogen, exhausted from CVD are reincorporated into the system after initial hydrocarbon generation (28). This strategic approach allows for the re-purification of the exhausted hydrocarbons, ensuring maximal utilization of the resources and preventing the buildup of contaminants within the system. This final phase of recycling the hydrocarbons exhausted from the CVD process, therefore, ensures the effective purification and use of product streams, exemplifying the reactor system's commitment to efficiency and sustainability.

### EXAMPLE 1

In one potential embodiment PI of the inventive system, shown in FIGS. 4 and 5, sodium oxide (Na₂O) serves as the sorbent, ruthenium (Ru) functions as the catalyst, and the system operates on an aluminum oxide (Al₂O₃) support. The reference numbers shown in FIG. 5 are for feed streams, process steps, and effluents that may be the same or similar to those of FIG. 2, and so are labeled with similar numbers to those in FIG. 2 but with 100 added; the similarities and differences between the feed streams, processes, and effluents of FIGS. 2 and 5 will be understood from this disclosure including this description of Example 1 and from the tables of information in FIGS. 2A and 5A. The reference numbers shown on FIG. 5 are described later in this document, in the table of FIG. 5A, and/or as follows in this paragraph. CO₂-lean air flowing from the reactor as effluent 116 during CO₂ capture, or H₂ and residual air flowing from the reactor as effluent 116 during reactor purification, may be purged in stream 222. Stream(s) 151 removes NH₃ and H₂O from the condenser system 150. Valve manifold 57, which feeds ultra-high purity CH₄ and H₂ to CVD 158. H₂ line 154 sends H₂ from the electrolyzer 110B to valve manifold 157 for combination with high purity CH₄ and H₂ stream 152 to reach the CVD operator's preferred final working concentration for the feed to the CVD unit 158. The CVD exhaust gas 170 may be sent to a recycle loop to recycle high purity methane and H₂ from said exhaust 170 to the processes 130, 140, 150 downstream of the reactor 120. In FIG. 5, exhaust gas 170 is shown being received by recycle loop manifold/piping 128 at the lower left and sent to recycle stream 128 at the top middle of the figure labeled “from CVD 170”.

A reactor 20 of a volume of 1 m³ containing the above dual function material initiates the process by drawing in ambient air (102) containing about 400 ppm CO₂ at a flow rate of 10 standard liters per minute (SLPM), modulated via mass flow controllers to ensure precision. The reactor is heated differentially across the bed of the reactor with the inlet (where pressure is highest) at near ambient temperatures and the bed of the reactor near the exhaust of the reactor (where pressure is lowest) heated to between about 20-50° C. based on the flow rate and pressure of the gas to differentially adsorb CO₂ containing carbon-12 over CO₂ containing carbon-13 based on natural phenomena such as active site specificity and kinetic isotope effects.

This gas stream is directed by the valve manifold (112) to contact the primary reactor (120A) until the Na₂O sorbent portion becomes saturated with CO₂, indicated by a CO₂ concentration sensor which monitors the exhaust stream (116A). Upon reaching a predetermined threshold of CO₂ breakthrough, the gas stream is redirected by the valve manifold (112) to a comparably equipped reactor (120B), while the original reactor (120A) undergoes a purge cycle (also discussed above as the “purification step”) to remove residual air. This purge cycle involves creating a slight vacuum within the reactor over a period of about 20 to 30 seconds at a pressure between about 10 and 100 torr at a temperature between about 15° C. and 60° C. The reactor is then purged with high-purity hydrogen at a flow rate of about 5 SLPM until at least five reactor volumes have flowed through the reactor (120A) under controlled conditions to ensure the substantially complete removal of residual air.

Next, the reactor (120A) continues to be subjected to a stream of high-purity hydrogen gas (108) at a flow rate of 5 SLPM, while the pressure within the reactor is maintained between about 700 torr and 800 torr and temperature between about 225° C. and 275° C. to ensure optimal catalyst reduction conditions. The pressure and temperature are carefully calibrated to favor the desorption of CO₂ containing carbon-13 while leaving CO₂ containing carbon-12 adsorbed. The temperature of the reactor is then systematically increased to between about 300° C.-350° C. to stimulate the simultaneous desorption of CO₂ from the dual-function material and hydrogenation of the CO₂. Once again, the temperature is finely calibrated to selectively convert CO₂ containing carbon-12 to CH₄ while leaving CO₂ containing carbon-13 either in its original form as CO₂ or catalyze its reduction to CO.

To confirm the progress and completion of the catalytic conversion, the reactor system employs a gas chromatograph mass spectrometry or commercially available syngas sensors that monitor the exhaust gas (116A) for the presence of methane. Once the monitoring system detects that the exhaust gas stream (116A) comprises predominantly methane, the flow is redirected from the recycle stream (118) to the primary product stream (126).

Finally, the product gas (126) is combined with exhausted gas (128) from the chemical vapor deposition process (CVD 158 via 170) and subjected to a series of downstream upgrading steps involving fine conversion of additional CO₂ by additional hydrocarbon catalysis (130), pressure swing adsorption or temperature swing adsorption (140) to eliminate any trace impurities, such as water, residual carbon dioxide, and nitrogen. The output (152) of these multiple series process reactors (130, 140, 150), a high purity methane stream in high purity hydrogen, is then diluted further with high purity hydrogen (154) so that the high purity methane is only 1%-10% methane in 90% to 99% hydrogen ready for utilization. The contaminants (non-methane and non-hydrogen) in this final feed stream (156) to CVD (158) are expected to be less than about 0.01% by volume. This entire process P1, therefore, is an example of a unique embodiment that represents an efficient, sustainable, and reliable method for capturing and converting carbon dioxide into ultra-high purity methane stream with enriched isotopic purity used for CVD.

### EXAMPLE 2

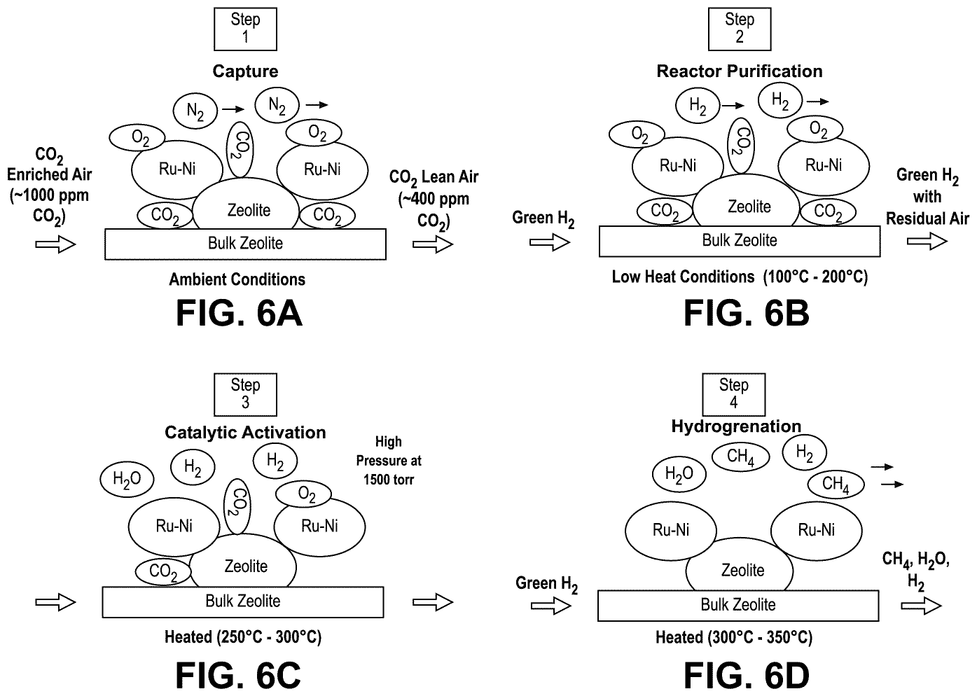

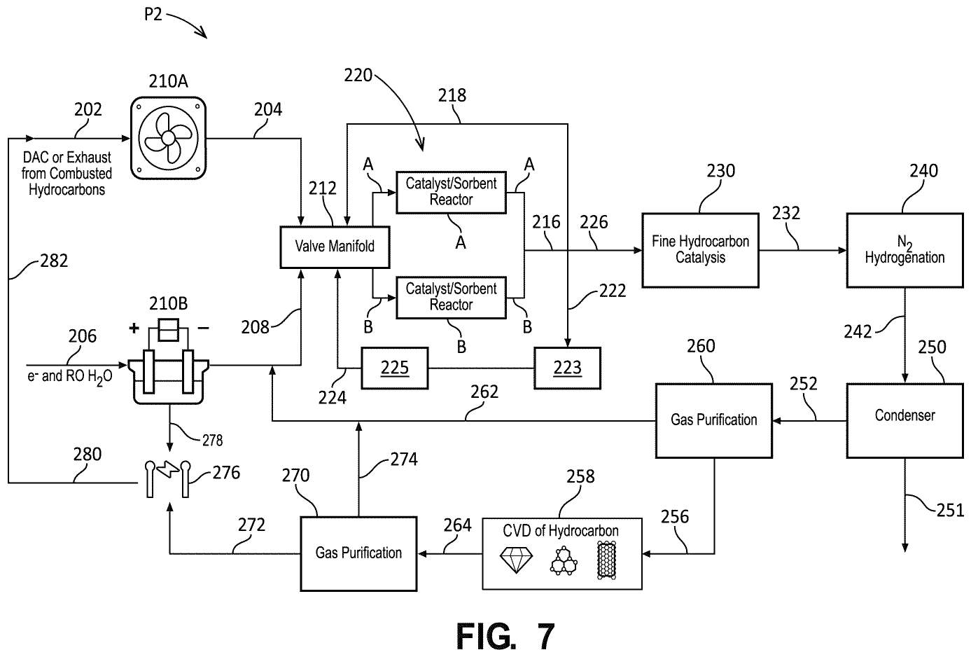

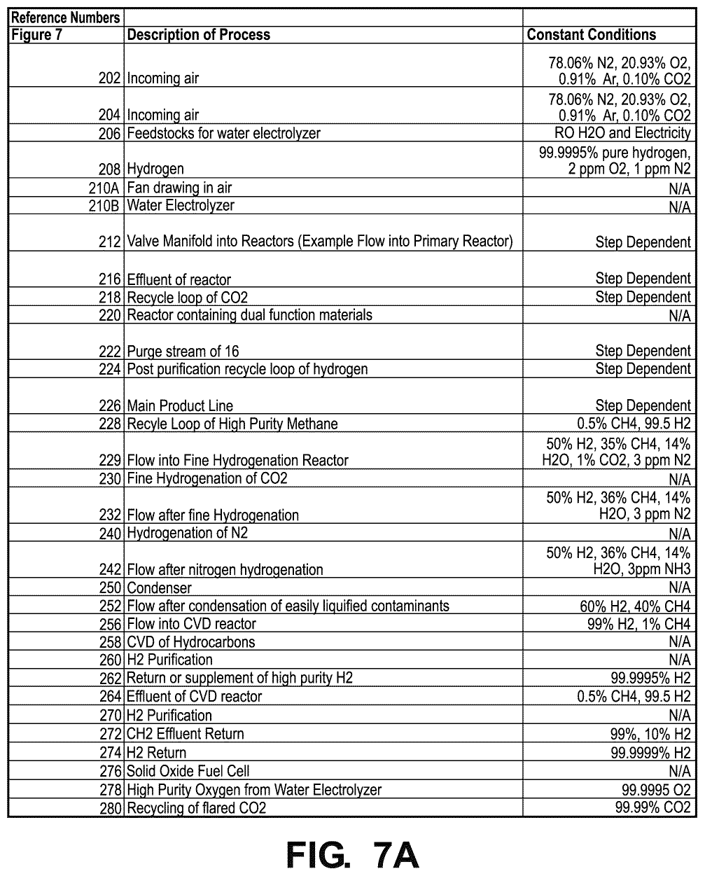

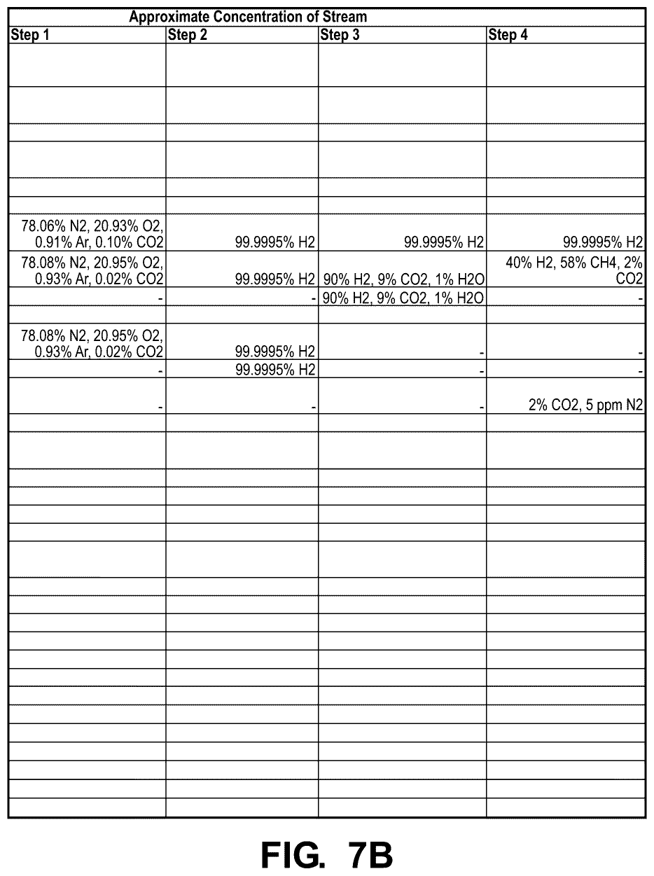

In another embodiment P2 of this inventive system, shown in FIGS. 6 and 7, nickel (Ni) and Ru are used as the catalyst, co-deposited on a zeolite substrate. The reference numbers shown in FIG. 7 are for feed streams, processes, and effluents that may be the same or similar to those of FIG. 2 or 5, and so are labeled with similar numbers to those in FIGS. 2 and 5 but with 100 and 200 added, respectively; the similarities and differences between the feed streams, processes, and effluents of FIG. 7 compared to those of FIGS. 2 and 5 will be understood from this disclosure including this description of Example 2 and from the tables of information in FIGS. 2A, 5A and 7A. The reference numbers shown on FIG. 7 are described later in this document, in the table of FIG. 7A, and/or as follows in this paragraph. CO₂-lean air flowing from the reactor as effluent 216 during CO₂ capture, or H₂ and residual air flowing from the reactor as effluent 216 during reactor purification, may be purged in stream 222. Valve manifold 223 is provided for receiving and then directing stream 222 to hydrogen purification 225 and then H₂ recycle via steam 224. Stream(s) 251 removes NH₃ and/or H₂O from the condenser system 250. Stream 252 feeds gas purification 260 that sends 90% ultra-high purity CH₄ via stream 256 to the CVD unit 258 and sends high purity H₂ back to the hydrogen line from the electrolyzer 210B for recycling to the reactor system via valve manifold 212. CVD exhaust/effluent 264 is sent to gas purification 270 to remove high purity H₂ from the CVD effluent, resulting in a CH₄ effluent 272 being sent with O₂ (278) to a solid oxide fuel cell 276, whereby CO₂ is recycled via stream 280 and stream 282 into the CO₂-containing gas of stream 202.

The reactor 220 of a volume of 1 m³ containing the above dual function material begins by mixing ambient air (206) containing CO₂ supplemented with excess CO₂ (280) from the exhaust of a solid oxide fuel cell (276) that is oxidizing excess hydrocarbons (272) from the effluent of a CVD reactor (264). The gas is cooled and then introduced (214A) into the reactor (220A) at a flow rate typically within the range of about 3-5 standard liters per minute (SLPM), controlled using mass flow controllers for accuracy.

The carbon dioxide laden gas stream passes through the valve manifold (212) to interact with the reactor (220A) that is uniformly at ambient temperature, in Step 1, until the zeolite sorbent portion becomes substantially saturated with carbon dioxide, which is determined by a carbon dioxide concentration sensor monitoring the exhaust stream (216A) or a reactor temperature sensor measuring any thermal events in the reactor occurring due to adsorption. Upon reaching a predetermined concentration at the reactor exit (216A) or the predetermined thermal cue, the carbon dioxide gas stream is redirected via the valve manifold (212) to another similarly equipped reactor (220B), while the initial reactor (220A) commences a purge cycle (Step 2) to eliminate residual air.

This purge cycle incorporates the generation of a slight vacuum in the reactor for about 30 seconds to 1 minute, at a pressure between about 5 and 20 torr. The reactor is then purged with high-purity hydrogen that comes both from water electrolyzers (210B) as well hydrogen purified from the main product gas (260) and CVD effluent (270). The hydrogen is introduced to the reactor at a flow rate of 2 SLPM, controlled to ensure the substantial removal of residual air. This cycle of vacuum and purge is repeated three times. The exhausted hydrogen (222) that is used to purge the reactor is sent to a recycle loop (224) that purifies out any contaminants using temperature swing adsorption and/or pressure swing adsorption to regenerate a hydrogen of greater than 99.99% purity before recycling it.

Following the purge cycle, the reactor is subjected to a hydrogen gas atmosphere at a pressure of about 1500 torr, without a continuous hydrogen flow. The reactor's temperature is then systematically increased to 275° C. to facilitate at least a portion of the reduction of the ruthenium and nickel catalysts.

Upon completion of the catalyst reduction (Step 3), the reactor temperature is brought to 325° C. and hydrogen flow (208) is resumed to facilitate the desorption of carbon dioxide from the dual-function material and catalytic conversion (Step 4) of the desorbed carbon dioxide into methane in the presence of the reducing hydrogen atmosphere.

A gas chromatograph or mass spectrometry unit continuously monitors the exhaust gas (216A) for methane. Once the exhaust gas stream (216A) is determined to consist mainly of methane, the flow is switched from 218 to the primary product stream (226) and to fine hydrogenation catalyst(s) (230). This reactor (230) can utilize similar catalytic materials for performing the CO₂ hydrogenation reaction as the main reactor (220) including nickel or ruthenium metal catalysts but may include additional catalysts such as cobalt, palladium, and rhodium among others that all have different temperatures for the methanation reaction. The fine hydrogenation reactor is at similar temperatures to the main reactor (˜300° C.) but could be at higher pressures (about 700-7000 torr) to ensure complete catalytic conversion of the carbon dioxide.

In this embodiment, after fine hydrogenation (230), the product gas undergoes an additional step of nitrogen hydrogenation (240). In this step, trace nitrogen is reacted with excess hydrogen to form ammonia at 1 atm of pressure or higher and at a temperature of at least 300° C., further enhancing the purity of the resulting methane.

The final product gas undergoes condensation and additional upgrading steps (250) involving pressure swing adsorption or temperature swing adsorption to eliminate any residual impurities, such as water, residual carbon dioxide, and the newly formed ammonia.

The resulting gas (252) then undergoes filtration with membrane gas separation technology (260) to reduce the concentration of hydrogen in the product stream to approximately 10%. The high purity hydrogen that is filtered/purified out is then returned (262) to the start of the process. Effluent from the CVD process (258) is then similarly filtered/purified (270), so that the hydrogen gas returns (274) to the initial hydrogen stream (208) and a mixture containing substantial amounts of CH₄ (272) is then oxidized via a hydrogen fuel cell (276) and the resulting CO₂ is used to supplement the carbon capture step (280).

The reactor system 220 and process P2 thus provide a high purity methane stream in 10% H₂ that can be directly used with further dilution with hydrogen before final CVD of the hydrocarbon (258). This entire process P2, therefore, is another example of a unique embodiment that represents an efficient, sustainable, and reliable method for capturing and converting carbon dioxide into ultra-high purity methane.

### EXAMPLE 3

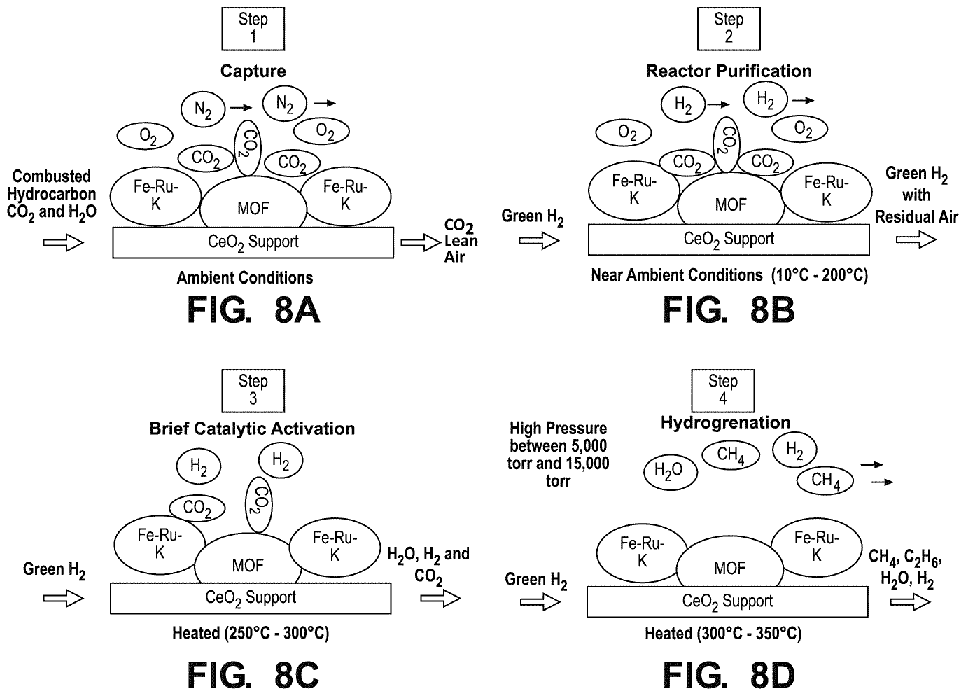

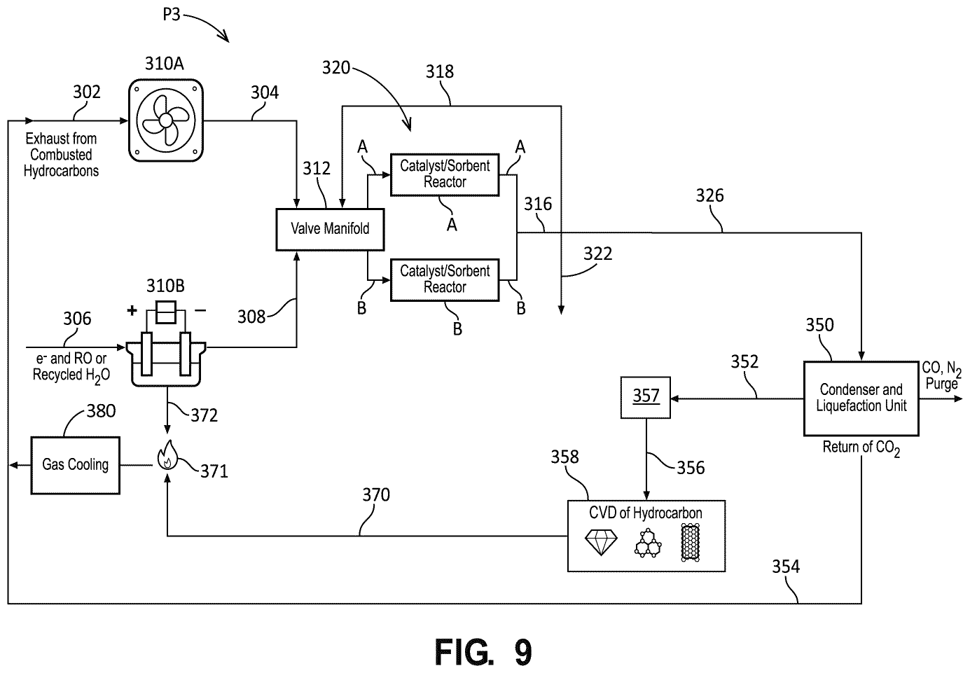

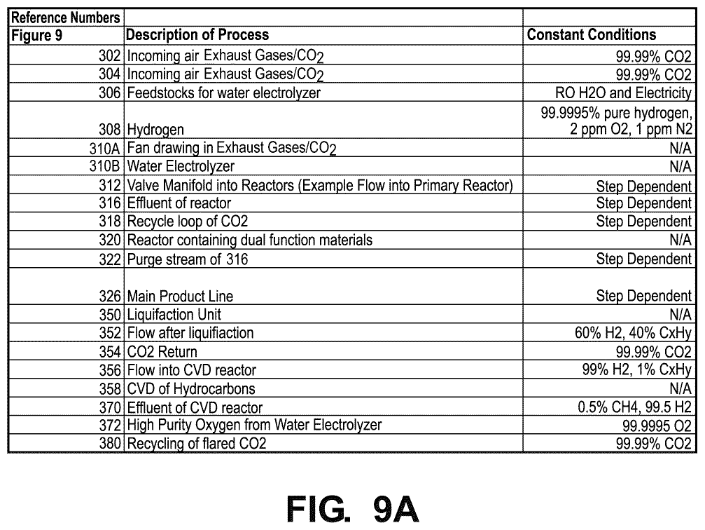

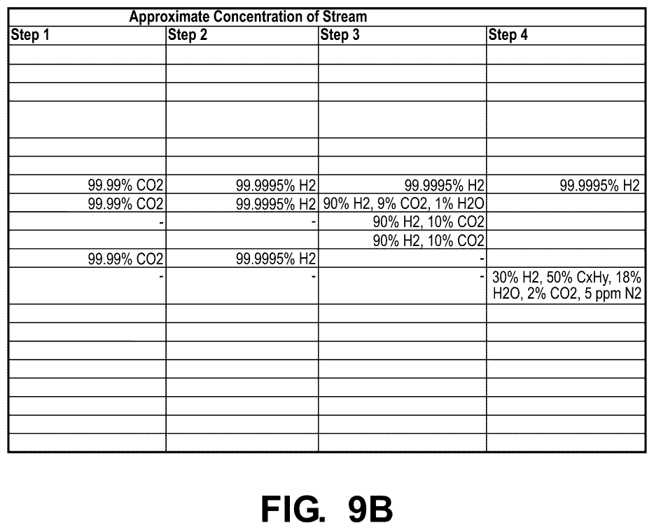

In a third illustrative embodiment of the invention, shown in FIGS. 8 and 9, a combination of iron, ruthenium, potassium (Fe—Ru—K) is used as the catalyst and is embedded into an amine-functionalized MOF, forming the dual-function material system potentially deposited on CeO₂ support and provided in main reactor 320. The reference numbers shown in FIG. 9 are for feed streams, processes, and effluents that may be the same or similar to those of FIG. 2, 5, or 7, and so are labeled with similar numbers but with 300 added to the numbers of FIG. 2; the similarities and differences between the feed streams, processes, and effluents of FIG. 9 compared to those of FIGS. 2, 5, and 7, will be understood from this disclosure including this description of Example 3 and from comparing the information in the table in FIG. 9A to FIGS. 2A, 5A and 7A.

Notably, in process P3, the initial gas stream (302) being fed into the reactor (320) is combusted hydrocarbon and or product gas derived directly from a CVD process. The water from the combusted hydrocarbons may be recycled into the electrolyzer (310B). Under the influence of a mass flow controller, the gas (304) containing a significant concentration of carbon dioxide is introduced into the reactor at a flow rate typically in the range of about 1 to 5 SLPM. The gas stream interacts with the reactor (320) until the MOF (metal-organic framework) sorbent portion becomes substantially saturated with carbon dioxide, as determined by a carbon dioxide concentration sensor located in the exhaust stream (316).

Upon reaching a predetermined concentration of carbon dioxide in the exhaust, indicating substantial sorbent saturation, the gas stream is redirected via a valve manifold (312) to a similarly configured secondary reactor (320B). Owing to the lean-oxygen conditions under which methane is combusted after the CVD process, there is only trace surplus oxygen in the incoming gas stream. Consequently, the Fe—Ru—K catalyst is not highly oxidized, thereby allowing the process to only include a brief catalytic activation or reduction step (Step 3) that will be accomplished as the system transitions from reactor purification to hydrogenation. Step 3 therefore may not require any dedicated time and instead any trace oxidation will be removed as the temperature is increased under hydrogen flow.

As the reactor (320A) proceeds to the hydrogenation step (Step 4), hydrogen flow of 1 SLPM is commenced and the pressure of the reactor is brought to between about 5,000 torr and 15,000 torr depending on the targeted composition of hydrocarbons, as higher pressure will typically produce more C—C bonds. Also, the temperature is adjusted to a suitable range, typically 300° C. and 450° C., initiating the desorption of carbon dioxide and catalytic conversion of the desorbed carbon dioxide into light hydrocarbons, primarily methane and ethane. This step is facilitated by the reduced Fe—Ru—K catalyst, embedded in the amine-functionalized MOF, which retains its reductive potential due to the absence of excess oxygen in the initial gas stream.

A gas chromatograph or a mass spectrometer monitors the exhaust gas stream (316) for the presence of hydrocarbons. When the exhaust stream contains predominantly hydrocarbons, the flow is switched to the primary product stream.

This product stream then undergoes liquefaction to separate out CO, CO₂, N₂, CₓH_{y}, and H₂. The trace N₂ and CO is purged while the carbon dioxide is recycled back through the reactor (354). The remaining gas is hyper pure light hydrocarbons in hydrogen. The process thereby delivers an ultra-high purity stream of methane and ethane (352), which could be reintegrated into the CVD process (358) or utilized for alternative applications. Note that, in FIG. 9, the CVD unit 358, being fed ultra-purity CₓH_{y} (typically CH₄) and H₂ (352) in stream 356 from CVD feedstock adjustment 357 (for example, adding hydrogen to dilute the CH₄), will exhaust effluent 370 that may be sent for combustion/oxidizing 371 with high purity O₂ from the electrolyzer 310B. This embodiment exemplifies an integrated and sustainable approach to carbon capture and methane production, particularly well-suited for processes such as CVD.

### EXAMPLE 4

In one potential embodiment of the inventive system, a substrate loaded with DFM-laden support for a reactor 420 is provided according to the following methods and compositions. A support layer between about 20 micrometers and 500 micrometers in average thickness of porous aluminum oxide (Al₂O₃) is first coated onto a FeCrAlY or FeCrAl, or similar alumina forming material when heat treated, flow-through monolith substrate with many parallel open channels coated with DFM. In a preferred embodiment, the alumina layer average thickness is between about 60 micrometers and 100 micrometers resulting in an alumina support loading of between 0.1 g/in3 and 2.5 g/in3 of flow-through substrate volume. Ruthenium (Ru) is then deposited on the alumina at a weight percent between 0.01% and 0.15% Ru resulting in a total loading of between 0.0007 g/in3 and 0.0105 g/in3 of flow through substrate. Nickel (Ni) is then deposited on the alumina at a weight percent between 0.1% and 5% Ni resulting in a total loading of between 0.007 g/in3 and 0.35 g/in3 per the flow through monolith substrate. A 1:2:0.5 mixture of sodium oxide (Na₂O), calcium oxide (CaO), and potassium oxide (K20) is then loaded onto the flow-through substrate at a weight percent between 5% and 15% sorbent resulting in a total loading of between 0.01 g/in3 and 2.5 g/in3 of flow-through substrate.

FIGS. 10 and 10A and B show reference numbers for the process P4 of this Example 4, which numbers are described in the table of FIG. 10A or in the following paragraphs. In use in Process P4, the reactor 420 with a 1 m3 interior reactor volume contains primarily the flow-through substrate with the support and dual function material, contained within the reactor wall. The process is initiated by performing multiple steps in the reactor: CO₂ capture, reactor discharge of non-sorbed species, and hydrogenation. After reactor discharge of non-sorbed species, temperature is raised, which activates the catalyst, begins desorption of CO₂, and starts hydrogenation.

The first step is started by drawing in ambient air (402, fan 410A) containing approximately 425 ppm CO₂ on average at a flow rate between about 10,000 standard liters per minute (SLPM) and 1,000,000 SLPM, modulated via mass flow controllers to ensure precision. In a preferred embodiment, air is drawn into the process at a flow rate between about 400,000 and 600,000 SLPM.

This gas stream (404) contacts the reactor 420 until the Na₂O sorbent portion becomes substantially saturated with CO₂, indicated by a CO₂ concentration sensor which monitors the exhaust stream. During this carbon capture step, CO₂-lean reactor effluent 416 may be purged through purge line 422. Upon reaching a predetermined threshold of CO₂, the gas stream is redirected to a comparably equipped reactor (not shown in FIG. 10 but understood from FIGS. 2, 5, 7, 9) or purged (422) in certain embodiments., while the original reactor 420 undergoes a second step purge cycle or “purification” to remove residual air. This purge cycle involves creating a slight vacuum within the reactor over a period of about 20 to 300 seconds at a pressure between about 0.1 torr and 100 torr at a temperature between about 15° C. and 60° C. In a preferred embodiment, the purge cycle begins by creating a slight vacuum over a period between about 180 seconds and 240 seconds at a pressure between about 0.5 torr and 1.5 torr. The reactor is filled with hydrogen, via stream 406 being fed to an electrolyzer 410B providing the hydrogen (408) to a pressure between about 1,000 torr and 50,000 torr. Preferably, the reactor is filled with hydrogen to a pressure between about 5,000 torr and 15,000 torr. The purge cycle repeats by creating a vacuum and then filling the reactor up to 5 times or more until the full removal of residual air. In a preferred embodiment, the reactor requires 2 to 4 vacuum-purge cycles to fully remove the air. Next, the reactor remains full of hydrogen at a pressure between about 1,000 torr and 50,000 torr. Preferably, the reactor remains pressurized with hydrogen between about 5,000 torr and 15,000 torr. Valves then open gas lines to a pump that circulates the hydrogen gas to the reactor. The pump circulates flow to the reactor between about 1 and 60 reactor volumes per minute. In a preferred embodiment, the pump circulates flow to the reactor between about 5 and 15 reactor volumes per minute. The temperature of the reactor is then systematically increased to 300° C. to stimulate the simultaneous desorption of CO₂ from the dual-function material and hydrogenation of the CO₂.

To confirm the progress and completion of the catalytic conversion, the reactor system may employ an infrared spectrometry and/or mass spectrometry unit, for the reactor effluent 416 or the recycle/recirculation loop 418 that may be operated during said temperature increase, to monitor the exhaust gas for the presence of methane. Once the monitoring system detects that substantially all of the hydrogen gas has been converted to methane, the reactor is evacuated with the product gas redirected to the primary product stream 426. A flow of hydrogen between about 1 SLPM and 50 SLPM is then introduced in the reactor for the remainder of the temperature ramp and temperature plateau. In a preferred embodiment, a flow of hydrogen between about 5 SLPM and 15 SLPM is introduced in the reactor 420 serving as a reactant to convert the adsorbed CO₂ to CH₄ and drive the product to the primary product stream 426. Once the reactor reaches 300° C., the reactor temperature is held at 300° C. until substantially all of the CO₂ is desorbed and converted to CH₄ with hydrogen. The molar ratio between the hydrogen introduced to the reactor during the heating step and the amount of CO₂ adsorbed should be held between about 1:4 and 1:8. In a preferred embodiment, the molar ratio between the hydrogen introduced to the reactor and CO₂ is between 1:4 and 1:4.1 resulting in an effluent directly from the core reactor that is greater than 90% by volume methane on a dry basis.

Finally, the product gas 426 is subjected to a series of downstream upgrading steps comprising condensation, pressure swing adsorption, membrane separation, reaction that might comprise a fuel cell to reduce the hydrogen composition, or temperature swing adsorption to eliminate or reduce trace impurities, such as water, residual carbon dioxide, hydrogen, and nitrogen. The downstream upgrading option shown in FIG. 10 is a fuel cell 450, which outputs water and electricity 454, and a stream 452 consisting, or consisting essentially, of high purity CH₄ with a desired amount of H₂. In this manner, the output of the reactor 420, an ultra-high purity methane stream, can be adjusted in hydrogen content by the fuel cell 450 and/or in a fine-tuning (457) of hydrogen content, and then be used (456) in chemical vapor deposition (CVD 458) or other processes either as a pure gas or diluted further in hydrogen. See FIGS. 10A and B for exemplary compositions of the streams shown in FIG. 10, both during constant conditions (FIG. 10A) and during Steps 1-4 (FIG. 10B) wherein Step 3 is the temperature ramping step described herein.

Reactor 420 in Process P4 may be the same or similar to reactor 850 shown in FIGS. 25 and 26. Reactor 850 is a flow-through reactor comprising multiple cylindrical reactor portions/walls 851 connected by bolted flanges 852. Air is drawn into air inlet 854 by air blower 860 and flows through the open channels of the monolith(s) in the interior of the reactor and exits at air outlet 858. At each end of the reactor are valves 856, 862, for example, gate valves, butterfly valves, or other large diameter valves. Recycle/recirculation pump 868 may be a pump, blower or other means for recycling the reactor gasses, for example, hydrogen, CO₂ and methane, from at or near the reactor outlet via high temperature valves 864, 872 and pipes 866, 870, and 874, to at or near the reactor inlet. This recycle loop may be further understood by referring to recycle loop 418 in FIG. 10. Six instrumentation ports 876 are provided on the right left sides of the reactor, for example, for thermocouples, pressure sensors, oxygen probes, gas sampling, or other specialty sensors. Hydrogen inlet ports 878 are provided at the bottom of each of the three reactor sections. Product evacuation ports 880 are provided at the top of each of the three reactor sections.

This entire process, therefore, embodies an efficient, sustainable, and reliable method for capturing and converting carbon dioxide into ultra-high purity methane used for chemical vapor deposition or other applications.

### EXAMPLE 5

In one potential embodiment of the inventive system, flow-through monolith with DFM is provided according to the following methods and compositions. A layer between 80 micrometers and 120 micrometers in average thickness of aluminum oxide (Al₂O₃) is deposited onto a cordierite honeycomb monolith flow-through substrate with 400 cells per square inch (CPSI). Ruthenium (Ru) is then deposited on the alumina support at a weight percent between about 0.6% and 0.8% Ru resulting in a total loading of between 0.05 g/in3 and 0.15 g/in3 of a flow through substrate. Sodium oxide (Na₂O) is then loaded or coated onto the flow-through substrate at a weight percent between about 7% and 12% Na₂O resulting in a total loading of between about 0.2 g/in3 and 0.3 g/in3 of flow-through substrate. The balance between the sorbent and catalyst has been set to ensure that the combination of the desorption of the CO₂ and the production of methane are balanced resulting in a process that requires minimal heating or cooling.

A single unit of the cylindric cordierite flow-through monolith with DFM is 42.5 mm in diameter and 50 mm in length. The DFM reactor piece is approximately 60 mL in volume. Thus, a reactor 520 consisting of four cordierite monoliths in series approximate a 240 mL reactor. Four cordierite pieces are loaded into a quartz tube approximately 400 mL in volume inside a tube furnace. The quartz tube is sealed by stainless steel flanges and fluorosilicone gaskets connected to a valve manifold on both sides. Downstream product gas is sampled with a residual gas analyzer with an in-line infrared CO detector. This gas sampling equipment is capable of measuring gas composition in the sub ppm regime as well as distinguishing isotopically enriched varieties of CH₄.

In use, the reactor 520 with a 240 mL interior reactor volume contains primarily the flow-through monoliths with the dual function material, contained within the reactor wall. The process is initiated by performing multiple steps in the reactor: CO₂ capture, reactor discharge of non-sorbed species, and hydrogenation. After reactor discharge of non-sorbed species, temperature is raised, which activates the catalyst, begins desorption of CO₂, and starts hydrogenation.

In use of the above-described reactor 520, a process P5 initiated by drawing in ambient air containing approximately 425 ppm CO₂ at a flow rate between about 50 standard liters per minute (SLPM) and 75 SLPM, modulated via mass flow controllers to ensure precision. The air is drawn in for about 60 minutes until the reactor is substantially saturated with CO₂ as measured by the mass spectrometer. During this CO₂ capture step, CO₂-lean air from the reactor effluent 516 may be purged through a purge line such as illustrated by line 522.

The reactor undergoes a purge cycle to remove residual air. This purge cycle involves creating a slight vacuum within the reactor over a period of about 30 to 180 seconds at a pressure between about 0.1 torr and 10 torr at room temperature. The reactor is filled with hydrogen to approximate atmospheric pressure between about 700 and 800 torr. The purge cycle repeats by creating a vacuum and then filling the reactor 3 or more times until the substantially full removal of residual air is achieved.

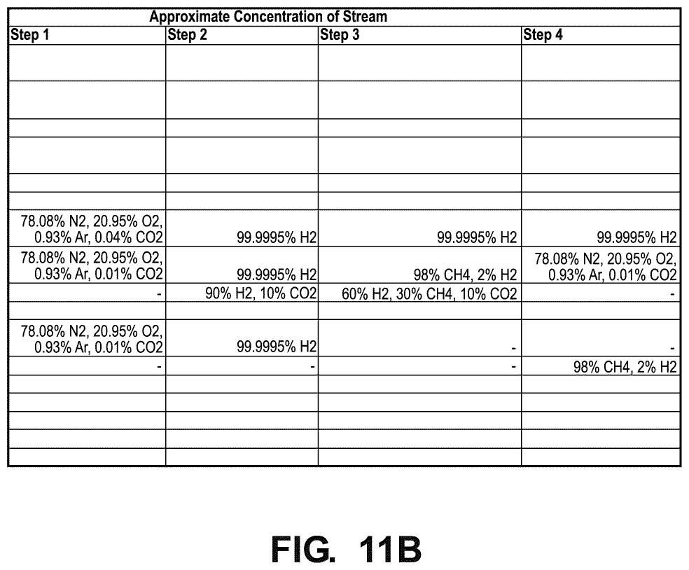

Next, the reactor remains full of hydrogen at near atmospheric pressure. Valves then open gas lines to a pump compatible with flammable mixtures that circulates the hydrogen gas within the reactor at a flow rate of up to about 1.5 SLPM. Thus, the recirculation pump circulates the flow to the reactor at approximately three reactor volumes per minute. The temperature of the reactor is then systematically increased to 300° C. at a ramp rate between about 5 and 200 C/min to stimulate the substantially simultaneous desorption of CO₂ from the dual-function material and hydrogenation of the CO₂. During this step, CO₂ containing reactor effluent 516 may be recycled to the reactor inlet by a recycle loop 518 such as portrayed in FIG. 11.

To confirm the progress and completion of the catalytic conversion, the reactor system may employ infrared spectrometry in addition to the mass spectrometry unit within the flow recirculation loop and reactor that continually monitors the exhaust gas for the presence of methane. Once the monitoring system detects that substantially all of the initial reactor volume of hydrogen gas has been substantially converted to methane, the reactor is evacuated with the product gas redirected to the primary product stream. Evacuation of the gas usually occurs between about 180° C. and 250° C. when the ruthenium catalyst is sufficiently active for the hydrogenation reaction. A flow of hydrogen between about 0.05 SLPM and 0.5 SLPM is continually introduced in the reactor serving as a reactant to convert the adsorbed CO₂ to CH₄ and drive the product to the primary product stream. Once the reactor reaches 300° C., the reactor temperature is held at 300° C. until substantially all of the CO₂ is desorbed and converted with hydrogen to CH₄. See FIGS. 11A and B for exemplary compositions of the streams shown in FIG. 11, both during constant conditions (FIG. 11A) and during Steps 1-4 (FIG. 11B) wherein Step 3 is the temperature ramping step.

Notably, during this stage the reactor requires less than 25 kJ of cooling or heating, as the endothermic desorption reaction and exothermic methanation reaction have been combined in order to approximate near thermoneutrality, as defined by a thermal gradient within the catalyst less than about 50° C. and more preferably between about 1 and 20° C. The molar ratio between the hydrogen introduced to the reactor during the heating step and the amount of CO₂ adsorbed should be held between about 1:8 and 1:35.

The product gas is cumulatively about 95% high purity hydrogen with about 5% high purity methane on a dry gas basis. The output of the reactor can then be used in chemical vapor deposition processes. This entire process, therefore, embodies an efficient, sustainable, and reliable method for capturing and converting carbon dioxide into ultra-high purity methane used for chemical vapor deposition or other applications.

Reactor 520 in Process P5 may be the same or similar to reactor 750 shown in FIG. 16. Reactor 750 comprises a cylindrical reactor wall 752 with endcaps 754 having current bus bars 756, current carriers/electrodes 760, and flow apertures 758 in fluid communication with the channels of the monolith inside the reactor. This reactor is an example of an open-ended channel monolith-containing reactor that is primarily flow-through, resulting in a relatively low pressure drop across the length of the reactor. One of the endcaps is for the positive electrode and the other for the negative electrode, each attached to wires or rods that run the length of the reactor. Thus, each channel has one wire/rod carrying current in each of the channel's four corners. Rods of the same polarity are diagonal from each other in each channel, which means opposite polarities exist across each channel wall. The positive and negative voltage across the wall will cause a dielectric-barrier discharge (DBD). The positive and negative electrodes allow electricity to be used to either directly heat some or all of the reactor or generate DBD discharge.

### EXAMPLE 6

In one potential embodiment of the inventive system, a flow-through monolith with DFM is provided according to the following methods and compositions. A support layer between 80 micrometers and 120 micrometers in average thickness of aluminum oxide (Al₂O₃) is deposited onto a FeCrAlloy monolith flow-through substrate with 600 cpsi. Ruthenium (Ru) is then deposited on the alumina at a weight percent between 0.6% and 0.8% Ru resulting in a total loading of between 0.05 g/in3 and 0.15 g/in3 of flow through substrate. Sodium oxide (Na₂O) is then loaded onto the flow-through substrate at a weight percent between 7% and 12% Na₂O resulting in a total loading of between 0.2 g/in3 and 0.3 g/in3 of flow-through substrate. The balance between the sorbent and catalyst has been set to ensure that the combination of the desorption of the CO₂ and the production of methane are balanced resulting in a process that requires minimal heating or cooling.

The FeCrAlloy flow-through monolith with DFM is cylindrical measuring 120 mm in diameter and 140 mm in length. The piece is approximately 1.5 liters in volume and placed immediately upstream of the reactor in a heating coil that is approximately 150 CPSI. The heating coil may be optionally coated with additional DFM material similar to the bulk flow-through substrate. The cylindrical reactor is sealed by welded stainless steel flanges connected to a valve manifold on both sides. Downstream product gas is sampled with a residual gas analyzer with an in-line infrared CO detector. This gas sampling equipment is capable of measuring gas composition in the sub ppm regime.

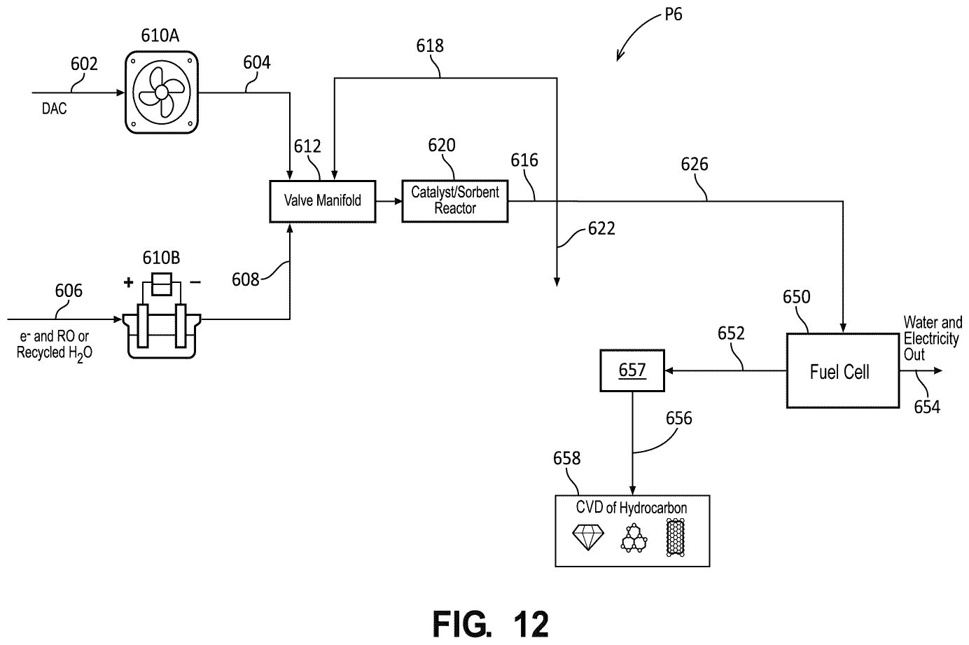

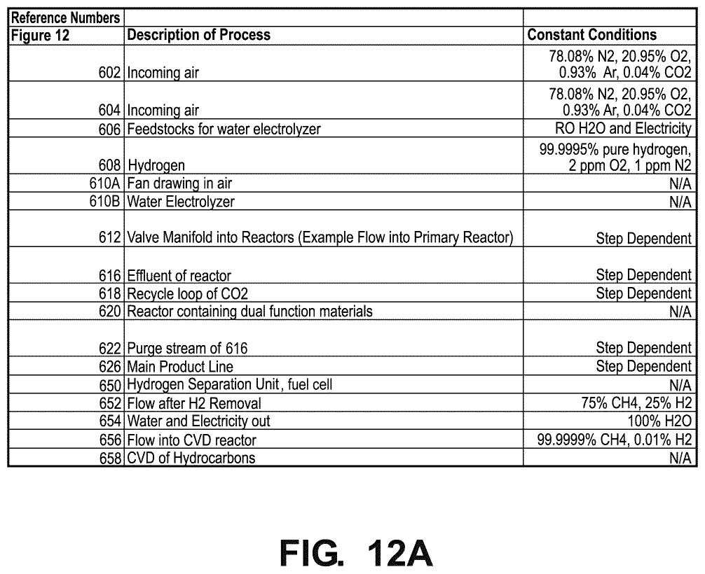

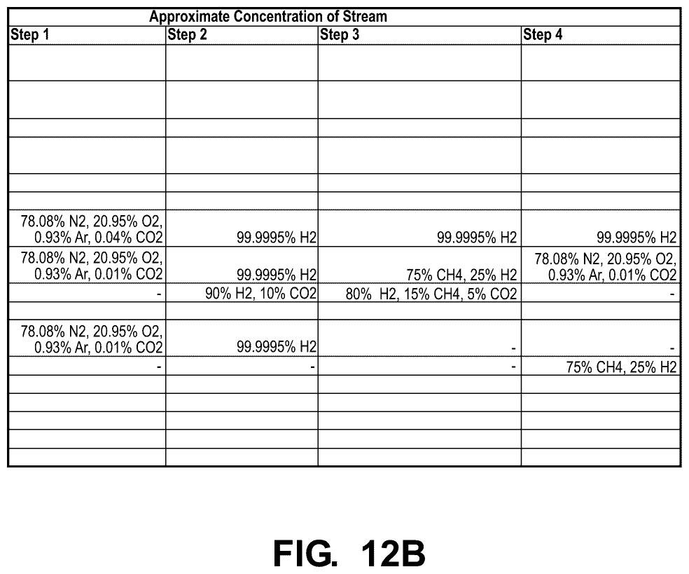

In use, as in process P6 shown in FIG. 12, the reactor 620 interior reactor volume contains primarily the flow-through monoliths with the dual function material described above, contained within the reactor wall. The process is initiated by performing multiple steps in the reactor: CO₂ capture, reactor discharge of non-sorbed species, and hydrogenation. After reactor discharge of non-sorbed species, temperature is raised, which activates the catalyst, begins desorption of CO₂, and starts hydrogenation.

The process initiates by drawing in ambient air containing approximately 425 ppm CO₂ at a flow rate between about 1000 standard liters per minute (SLPM) and 1500 SLPM, modulated via mass flow controllers to ensure precision. The air is drawn in for about 60 minutes until the reactor is substantially saturated with CO₂ as measured by the mass spectrometer or other methods. During this CO₂ capture step, CO₂-lean air from the reactor effluent 616 may be purged through a purge line such as illustrated by line 622.

The reactor undergoes a purge cycle to remove residual air. This purge cycle involves creating a slight vacuum within the reactor over a period of about 30 to 180 seconds at a pressure between about 0.1 torr and 10 torr at room temperature. The reactor is filled with hydrogen to approximate atmospheric pressure between about 700 and 800 torr. The purge cycle repeats by creating a vacuum and then filling the reactor 2 or more times until the substantially full removal of residual air.

For the next step, the reactor remains full of hydrogen at near atmospheric pressure. Valves then open gas lines to a pump that circulates the hydrogen gas with any product within the reactor at a flow rate of up to about 30 SLPM. Thus, the recirculation pump circulates flow back through the reactor at approximately 20 reactor volumes per minute. The temperature of the reactor is then systematically increased to about 300° C. at a ramp rate from about 5 to 200° C./min to stimulate the substantially simultaneous desorption of CO₂ from the dual-function material and hydrogenation of the CO₂ to methane. During this step, CO₂ containing reactor effluent 616 may be recycled to the reactor inlet by a recycle loop 518 such as portrayed in FIG. 12.

To confirm the progress and substantial completion of the catalytic reaction, the reactor system may employ infrared spectrometry in addition to the mass spectrometry unit within the recirculation loop and reactor that continually monitors the exhaust gas for the presence of methane. Once the monitoring system detects that substantially all of the initial reactor volume of hydrogen gas has been converted to methane, the reactor is evacuated with the product gas redirected to the primary product stream. Evacuation of the gas usually occurs between about 180° C. and 250° C. when the ruthenium catalyst is sufficiently active for the hydrogenation reaction. A flow of hydrogen between about 0.05 SLPM and 0.5 SLPM is continually introduced in the reactor serving as a reactant to convert the adsorbed CO₂ to CH₄ and drive the product to the primary product stream. Once the reactor reaches about 300° C., the reactor temperature is held at 300° C. until substantially all of the CO₂ is desorbed and converted to CH₄. See FIGS. 12A and B for exemplary compositions of the streams shown in FIG. 12, both during constant conditions (FIG. 12A) and during Steps 1-4 (FIG. 13B) wherein Step 3 is the temperature ramping step.

Notably, during this stage the reactor requires less than 25 kJ of cooling or heating as the desorption reaction and methanation reaction have been carefully calibrated in order to approximate thermoneutrality as defined by a gradient less than about 50 C. The molar ratio between the hydrogen introduced to the reactor during the heating step and the amount of CO₂ adsorbed should be held between about 4:1 and 6:1 (hydrogen: CO₂).

The product gas is cumulatively about 5% high purity hydrogen with about 95% high purity methane on a dry gas basis. The output of the reactor can then be used in chemical vapor deposition or other processes. This entire process, therefore, embodies an efficient, sustainable, and reliable method for capturing and converting carbon dioxide into ultra-high purity methane used for chemical vapor deposition or other processes.

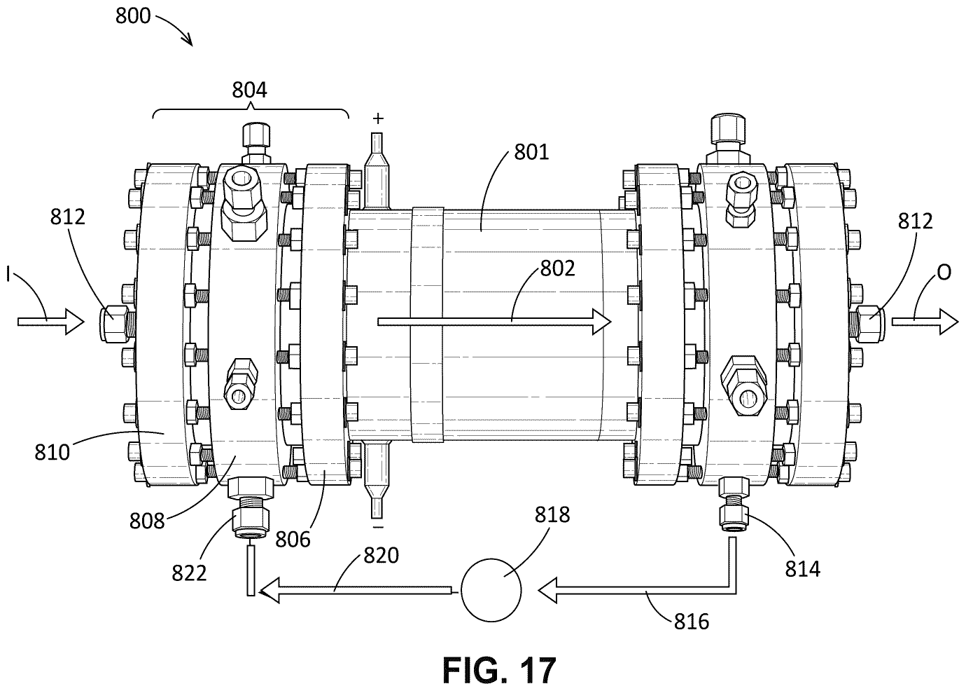

Reactor 620 in Process P6 may be the same or similar to flow-through reactor 800 shown in FIGS. 17-24. Reactor 800 comprises cylindrical reactor wall 801 with a flange assembly 804 at each end comprising inner 806 and outer 810 flanges and a port flange 808 between flanges 806 and 810. Flow 802 comprises inlet flow I, through the reactor, that is, through the open-ended channels of the monolith comprising DFM contained within the reactor interior space, to outlet O, via inlet and outlet fittings 812. Note that the outlet fitting 812 may serve at the vacuum port during the reactor purification step. The port flanges 808 provide ports for hydrogen and sensors, and dedicated ports 814, 822 for recycling reactor effluent from fitting 814, through lines 816 and 820, by means of pump/blower 818. Thus, the reactor effluent may be pulled back to the inlet of the reactor, as will be understood from the discussion of recycle/recirculation elsewhere in this document and by referring to recycle loop 618 in FIG. 12.

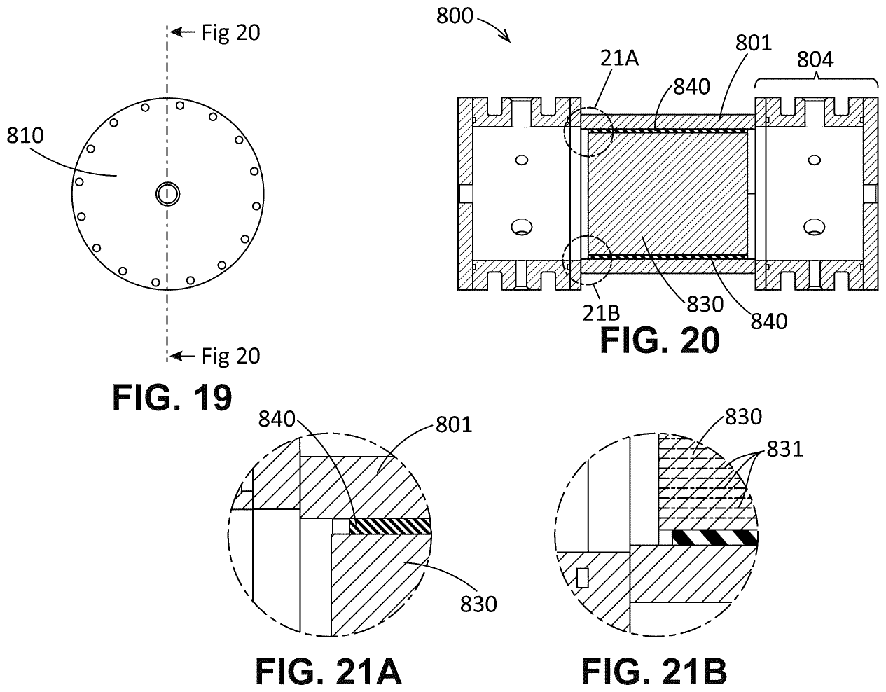

Further regarding reactor 800, an end view of the reactor is shown in FIGS. 19 and 22, and the reactor interior is shown in the longitudinal cross-sections in FIGS. 20 and 23. As shown in FIGS. 20 and 23, the interior of the two reactor-end flange assemblies 804 are spaces for inlet and outlet flow of gas (ports being visible in the inner surface of the assemblies 804) and the interior of the reactor between the assemblies 804 is substantially completely filled with the monolith comprising DFM 830. The monolith with DFM 830 comprises many longitudinal open-end channels for flow of gas and for contact with the DFM, as will be understood from this document, but those channels do not show in FIG. 20 figure due to the small size of the channels. However, an enlarged, detail view of the area of the reactor circled in FIG. 20 and labeled 21B shows with schematic dashed lines the open-ended flow channels 831 through the monolith substrate. It will be understood from this disclosure that the open channels extend longitudinally all along the length of the monolith, and both ends of each the channel 831 being open for accepting fluid flow into the channel at one end, allowing flow through the entire length of the channel, and out of the channel at the other end.

FIGS. 20 and 21 show a first exemplary method of sealing the monolith to the reactor wall 801, to avoid bypass of gasses around the monolith. FIG. 20 shows a layer of sealant 840 extending along preferably the entire length of the monolith 830 and understood to extend all around the outer circumference of the monolith 830. In an alternative method of preventing gas bypass around the monolith, FIGS. 23 and 24 show an O-ring 845, near the end-face of the monolith, extending around the entire circumference of the monolith to seal the monolith 830 to the reactor wall 801.

### EXAMPLE 7

In one potential embodiment of the inventive system, a reactor with the same nominal dimensions and composition of DFM as in Example 5 is employed in a continuous mode to upgrade CO₂-rich streams with near-thermoneutral performance. As in Example 6, the balance between the sorbent and catalyst has been set to ensure that the combination of the desorption of the CO₂ and the production of methane are balanced resulting in a process that requires minimal heating or cooling.

The reactor is heated to 240° C. and held at this temperature with an external heating coil and thermocouple assembly that regulates the temperature. A stream of pure CO₂ flowing at 0.2 LPM is combined with a stream of pure hydrogen produced via water electrolysis flowing at 0.8 LPM such that the two reactants are at a stoichiometric ratio for the methanation reaction. The CO₂ stream is outfitted with a switching valve that can divert Argon flowing at 0.2 LPM into the reactor instead of CO₂. An additional thermocouple is placed into the reactor on the face of the monolith on the downstream side. While maintaining a constant flow rate of H₂, the switching valve that can select between Ar and CO₂ is activated. Thus, while Ar is flowing alongside H₂ no methanation is possible and the reactor will settle back to the setpoint temperature of 240 C. However, when CO₂ is allowed into the reactor alongside H₂ at a stoichiometric ratio, methanation proceeds with ideally over 70% conversion and the temperature in the reactor changes in response. To confirm the progress of the catalytic reaction, the reactor system may employ infrared spectrometry in addition to a residual gas analyzer unit that continually monitors the exhaust gas for the presence of CH₄, CO₂, Ar, and H₂. Once the monitoring system detects that the continuous conversion of H₂ and CO₂ to CH₄ has reached a steady state, or that the Ar saturation within has reached a steady state, the Ar/CO₂ switching valve is actuated (see “regeneration” in FIGS. 13B and 14). Due to the presence of a sorbent subunit in the dual function material coating, the exothermic reaction of CO₂ methanation is partially counterbalanced by adsorption/desorption of CO₂ and other site-specific reactions that are not present in a traditional catalyst system. As a result, the temperature fluctuations in the reactor are within about 10° C. and indicate a slight temperature decrease during methanation and a slight increase while under Ar, possibly due to the reduction of the catalyst material and other complex surface reactions.

This process is schematically portrayed as process P7 in FIG. 13, wherein the reactor 720 interior reactor volume contains primarily the flow-through monoliths with the dual function material described above, contained within the reactor wall. Feedstocks for the reactor 720, supplied via valve manifold 712, are CO₂ feed 702A through line 704, argon feed 702B through line 706, and hydrogen 702C through line 708. Reactor effluent 726 may be monitored by mass spectrometry 730 prior to flowing downstream, for example in the laboratory environment to purge line 736. See FIGS. 13A and B for exemplary compositions of the streams shown in FIG. 13, during constant conditions (FIG. 13A) and during methanation and regeneration (FIG. 13B).

FIG. 14 illustrates the near thermoneutral dynamics of methanation of a sorbent-enhanced catalytic converter, operated similarly to Example 7. Though conventional wisdom would expect a large temperature increase due to the exotherm of methanation (see FIG. 15), the results shown in FIG. 14 illustrate the surprising results from the combination of sorbent and catalyst, wherein methane-production is accomplished with limited to no exotherm with a temperature change less than about 20° C.

### EXAMPLE 8

In one potential embodiment of the inventive system, a gas with significant hydrogen and methane content such as the effluent from the core reactor in Example 4, Example 5, or Example 6 is directed to the anodic compartment of an electrochemical device as illustrated in FIG. 27 (initial electrochemical device was part number FCS-C1000 as sourced from Horizon Fuel Cells America). The anode is composed primarily of a platinum-nickel alloy. The anode forms an electrochemical connection with a cathode that consists of a nickel plate though may consist of other metals alloyed with nickel. The cathode and anode are separated by at least one ionomer layer that allows for the exchange of ions.

Referring to FIG. 30, initially the incoming gas stream consists of 95% H₂ and 5% Ar and is flowed through the hydrogen fuel cell disconnected from any load. The fuel cell increases the pressure in the anodic compartment to between 15-150 PSI and either runs in a steady state condition or in batch mode regularly refilling the hydrogen content in the anode. The fuel cell registers the flow of hydrogen with an elevated open circuit potential. This gas mixture (FIG. 30) serves as a comparison for the later mixture of hydrogen and methane (FIG. 31).

The fuel cell is then connected to an electrical load either in the form of a power resistor or an electronic device that is carefully calibrated to provide a power draw that is equal to the power content of the incoming hydrogen oxidation target. The output of the anodic compartment is measured and the results from the embodiment are included in FIG. 30. Within seconds of applying a load to the fuel cell, the hydrogen content in the exhaust of the anodic compartment significantly decreases. The content of the fuel cell approximates 99.99% Ar in 0.01% H₂. Similarly, when the experiment is run with an incoming gas stream of 95% H₂/5% CH₄, the exhaust stream approximates a composition of 99.9% CH₄/0.1% H₂ (FIG. 31).

This embodiment highlights the surprising result that by carefully calibrating the identity of the fuel cell components and power load, nearly all hydrogen can be consumed in a heterogeneous gas mixture comprising hydrogen and methane. This approach may be valid for streams produced from processes discussed herein when a reduced hydrogen feedstock is desired for a downstream user, for example, a CVD process preferring a low-hydrogen feedstock. The inventors believe that this is the first instance of fuel cell(s) being used to selectively oxidize hydrogen in a methane/hydrogen stream for purification, especially after a methanation reactor. Ethane Production:

Ethane may be beneficial or desired in CVD when manipulating the kinetics of the CVD reaction or when aiming to manipulate the properties of the product made via CVD. It may be noted that the proportions of methane and ethane produced from certain embodiments may be controlled mainly by pressure, catalyst, and sorbent composition. Pressure and sorbent composition affect the local concentration of carbon potentially leading to creating carbon-carbon bond formation. Catalysts with higher percentages of iron or cobalt or rhenium will tend to catalyze carbon-carbon bond formation.

### Reactor Size and Configuration

The processes described in this document may be performed in differently sized reactors including research and development reactors and/or pilot-plant reactors, such as those portrayed in the Figures, and larger commercial-scale reactors. Commercial-scale reactors will likely contain many monoliths each between 6 inches and 12 inches in length. For example, based on an expected individual monolith length of 9-12 inches, a 12-foot reactor may contain as many as 15 monoliths. While flanges of reactors shown herein may, in certain embodiment, be adapted for use in adding or removing heat from the reactor, certain embodiments of the invention are expected not to need such adaptation or any other equipment for heating or cooling of the reactor because the reactions in the reactor are near thermoneutral. Therefore, certain embodiments need no flanges and/or comprise no heat exchanger for removing heat from the reactor being in thermal communication with the reaction chamber.

### Summarizing Certain Embodiments

Certain embodiments of the invention may be summarized generally as encompassing novel methods, systems, and materials for the efficient capture of carbon dioxide and its conversion into ultra-high purity hydrocarbons, particularly methane. In various embodiments, the methods comprise the following steps: utilizing a reactor equipped with both a sorbent and catalyst; initiating contact between a gas stream containing carbon dioxide and the reactor until the sorbent portions reach a state of substantial saturation with carbon dioxide; diverting the stream of gas comprising carbon dioxide to a substantially similar parallel reactor; removing the residual air via vacuum and/or purge cycles with high-purity gas; introducing a stream of hydrogen gas to the low pressure reactor containing the carbon dioxide-saturated sorbent and catalyst potentially to a pressure above ambient; increasing the temperature to facilitate the desorption of carbon dioxide from the dual-function material; and catalytically transforming the carbon dioxide with the hydrogen gas into methane. Notably, the adsorption and desorption of carbon dioxide occur within the sorbent portions, while the reaction of carbon dioxide with hydrogen gas to yield methane takes place at a temperature substantially consistent with the desorption stage on the catalytic unit. Finally, the product gas undergoes downstream upgrading steps to remove or reduce impurities of water, carbon dioxide, and nitrogen. This invention represents a significant advancement in carbon capture and conversion processes, offering a highly efficient and reliable method for producing ultra-high purity methane for processes such as, but not limited to, chemical vapor deposition.

From the above description and figures, it will be understood that certain embodiments of the invention may comprise, consist essentially of, or consist of, catalyst, sorbent, and support formulation(s), and/or methods of synthesizing the formulation(s), that are specially adapted for effective performance during frequent high/low-pressure switching, for example, including vacuum/purge cycles. In preferred embodiments, said formulations are dual functional materials (DFM). Certain embodiments comprise processes for carbon dioxide capture and conversion to high purity methane and/or ethane that comprise using said formulation(s) and comprising said frequent high/low-pressure switching and vacuum/purge cycles. Certain embodiments of the formulations and processes are specially adapted for effective performance at a range of pressures from about 0.001 torr to about 25,000 torr.

Certain of these embodiments are adapted to capture CO₂ from air and to produce a feed stream to a chemical vapor deposition process that contains hydrogen, methane and/or ethane, and less than about 1 ppm nitrogen. Certain of these embodiments are adapted to capture CO₂ from air and to produce a feed stream for said chemical vapor deposition processes that contains hydrogen, methane and/or ethane, and less than about 1 ppb nitrogen or from about 0.1 ppb to 10 ppm nitrogen.

In certain embodiments, the carbon dioxide capture and conversion to high purity methane and/or ethane comprises a dual functional material used in a single reactor instead of having carbon capture and methanation in separate reactors/processes. In certain embodiments, the processes/materials described herein may further comprise novel purification, of the effluent of the DFM, by one or more recycle/input streams to improve environmental impacts, process efficiency and/or effectiveness, and/or to customize process effluents for particular CVD processes. Certain of the processes/materials described in this paragraph and the preceding two paragraphs may comprise methods for heating the DFM reactor in a way that maximizes CO₂ capture and subsequent conversion to hydrocarbon and reactor efficiency. Certain embodiments processes/materials described in this paragraph and the preceding two paragraphs may comprise managing temperature, pressure, and flow rates to create isotopically pure methane.

In view of this disclosure and the preceding three paragraphs, it will be understood that certain embodiments will not comprise carbon capture and hydrogenation equipment and processes that are entirely separate from each other, for example, in separate reactors and/or in separate facilities. Further, it will be understood that certain embodiments do not include conventional DFM materials that are adapted substantially or entirely only for near-ambient pressure. Further, it will be understood that certain embodiments do not include a nitrogen purge of any DFM reactor, and especially certain embodiments do not include any purge of any DFM reactor that contains 80% or more nitrogen.

From the above description and the Figures, it also will be understood that certain embodiments may comprise, consist essentially of, or consist of one or more of the following items:
1) A process for making methane from CO₂ and hydrogen comprising providing a catalyst and a solid sorbent together in a reactor chamber wherein the process is cyclic and comprises a sorption step for capturing CO₂ with the solid sorbent, a vacuum step operating with a sub-atmospheric pressure less than 1 bar absolute to discharge non-sorbed species from the reactor chamber, and a conversion step wherein hydrogen is fed to the reactor chamber to produce methane with the CO₂ previously sorbed in the sorption step by hydrogenation, wherein all three steps occur in the same reactor chamber displaced in time. The process of item 1 above wherein no heat exchanger for removing heat from the reactor is in thermal communication with the reaction chamber. The process of item 1 above wherein reactor chamber effluent after the vacuum step comprises less than 10 ppm nitrogen. The process of item 1 above wherein reactor chamber effluent after the vacuum step comprises less than 1 ppm nitrogen. The process of item 1 above wherein the sorption cycle operates at less than 50° C. and the conversion step operates at a temperature greater than 180° C. The process of item 1 above wherein reactor temperature is raised from a vacuum step temperature to a beginning conversion step temperature, and, during said hydrogenation, the reactor temperature increases less than 20° C. The process of item 1 above wherein reactor temperature is raised from a vacuum step temperature to a beginning conversion step temperature whereby said hydrogenation begins, and, as hydrogenation continues, the reactor temperature decreases. The process of item 1 above wherein a preheater selected from a group consisting of an electric and a Joule heating exchanger is provided upstream of the reactor chamber to preheat a gas stream comprising hydrogen prior to flowing into the reactor chamber containing the catalyst and sorbent. The process of item 1 above wherein said sub-atmospheric pressure is achieved using a vacuum pump. The process of item 1 above wherein the catalyst and sorbent are coated on walls of open-ended flow channels of a catalyst substrate received in the reactor chamber, and, for example, the catalyst may comprise Ru and/or the sorbent may comprise Na or Ca, and in certain embodiments the catalyst and solid sorbent and a porous support are a dual function material coating on the support as coated on the substrate and the sorbent is between 1 and 20 wt % of the coating. In certain embodiments described above in this paragraph, a reactor chamber effluent, during at least a portion of a single pass of said hydrogen being fed to the reactor chamber in the conversion step, is methane gas with a purity of greater than 90% on a dry basis. In other embodiments of this paragraph, a reactor chamber effluent is recirculated to the reactor chamber during at least a portion of said conversion step and the process produces a reactor chamber product steam that is methane gas with a purity of greater than 90% on a dry basis.
2) A process for making methane from CO₂ and hydrogen, the process comprising providing a catalyst and sorbent provided in a reactor with a reactor length, the catalyst and sorbent being in contact for at least a portion of the reactor length, the process further comprising CO₂ being captured from a stream comprising the CO2 and N₂ at an inlet N₂ concentration, and subsequently feeding a hydrogen stream to the reactor so that the captured CO₂ is hydrogenated to methane and a methane reactor effluent is produced, wherein the methane effluent contains an exit N₂ concentration less than 10 ppm N₂ and the exit N₂ concentration is lower than the inlet N₂ concentration. The process of item 2, wherein the methane reactor effluent contains less than 1% CO₂, and the reactor effluent is recirculated to the reactor during at least a portion of said feeding of a hydrogen stream to the reactor.
3) A method for making a combined sorbent and catalyst coating on interior surfaces of an open flow channel reactor body wherein the reactor body is made from an alumina forming material and the maximum flow channel opening is less than 2 mm in width and/or in height in at least a portion of the flow length. The method of item 3 wherein the reactor body is heat treated to at least 800 C before coating the combined sorbent and catalyst onto said interior surfaces.
4) A process for making a hydrocarbon product from CO₂ and hydrogen using a reactor body comprising a sorbent and catalyst wherein the process comprises discontinuous flow of hydrogen to said reactor body, the flow of hydrogen occurring during a hydrogenation portion of the process wherein the temperature change within said reactor body is less than 20 C rise during said hydrogenation portion of the process. The process of item 4 where the hydrocarbon product is methane, so that said hydrogenation portion of the process comprises reaction of CO₂ and said hydrogen.
5) A method for producing high purity methane from a gaseous stream comprising CO₂, the method comprising providing a cellular reactor having a length, and performing a step wherein the gaseous stream flows past a dual function material (DFM) material, on walls of a plurality of open-ended cells of the cellular reactor, along at least a portion of the reactor length, so that CO₂ is adsorbed, and performing a step comprising providing hydrogen in the reactor so that the adsorbed CO₂ and hydrogen are converted to methane and wherein produced methane gas is a high purity methane with a purity of greater than 90%. The method of item 5 further comprising an additional step prior to said conversion to methane gas, the additional step comprising, after said CO₂ is sorbed, purging the reactor of non-sorbed species from the gaseous stream, the non-sorbed species comprising nitrogen. In the method immediately above in this paragraph, the method may be a cyclic process wherein said gaseous stream comprising CO₂ flowing past the DFM so that CO₂ adsorbs, said purging, and said CO₂ and hydrogen converting to methane are repeated sequentially multiple times; in certain embodiments of this method, purity of the produced methane is greater than 95%, or purity of the produced methane is greater than 98%, and/or the concentration of nitrogen in the high purity methane is less than 1%, less than 1%, less than 0.1%, less than 10 ppm, less than 2 mm, or less than 1 ppm.
6) A process for adjusting hydrogen content of a dual function material (DFM) effluent gas comprising hydrogen and gaseous hydrocarbons from a cyclic process producing the hydrocarbons over DFM, the process comprising using a hydrogen fuel cell on said DFM effluent gas and providing a fixed or variable electrical load, to alter the concentration of hydrogen in a fuel cell effluent gas, exiting the fuel cell, compared to said DFM effluent gas. The process of item 6 where the hydrocarbon is methane. The process of item 6 where hydrogen concentration in the fuel cell effluent gas is less than 10%. The process of item 6 where hydrogen concentration in the fuel cell effluent gas is less than 1%.
7) A process for using a reactor containing dual functional material, the reactor having two ends and a length between the two ends, the process comprising depositing current via at least one electrode to at least one of said two ends to allow said current to reach at least a portion of the reactor length where reactor feedstock flow traverses the length of the reactor, wherein at least one power source is provided outside the reactor and the at least one electrode is directly connected with the power source and spans at least a portion of the length of the reactor.
8) The process of any of the above items or subitems, wherein the reactor and the DFM-holding monolith within the reactor are adapted so that pressure drop through the reactor via the monolith channels, during CO₂ adsorption, is less than 1 bar or less than 10,000 Pa, preferably less than 1,000 Pa, and more preferably less between 200 and 1,000 Pa.
9) The process of any of the above items or subitems, for converting a plurality of reactants to at least one product in a reactor body comprising a catalyst and adsorbent in intimate contact within the reactor body wherein the adsorbent preferentially adsorbs a target reactant to increase the conversion of the target reactant in a single pass through the reactor so said conversion above the per-pass conversion of the target reactant that occurs in the absence of a sorbent under substantially similar process flows of the target reactant and similar temperature, pressure, catalyst composition and catalyst loading. As described herein, therefore, CO₂ is an example of said target reactant, wherein adsorption of CO₂ by the sorbent increases conversion of CO₂ to methane compared to the per-pass conversion that would be obtained if there were no capture of the CO₂ by a sorbent.

Although this disclosed technology has been described above with reference to particular means, materials and embodiments, it is to be understood that the disclosed technology is not limited to these disclosed particulars but extends instead to all equivalents within the broad scope of the following claims.

1. A process for making methane from CO₂ and hydrogen comprising providing a catalyst and a solid sorbent together in a reactor chamber wherein the process is cyclic and comprises a sorption step for capturing CO₂ with the solid sorbent, a vacuum step operating with a sub-atmospheric pressure less than 1 bar absolute to discharge non-sorbed species from the reactor chamber, and a conversion step wherein hydrogen is fed to the reactor chamber to produce methane with the CO₂ previously sorbed in the sorption step by hydrogenation, wherein all three steps occur in the same reactor chamber displaced in time.

2. The process of claim 1 wherein no heat exchanger for removing heat from the reactor is in thermal communication with the reaction chamber.

3. The process of claim 1 wherein reactor chamber effluent after the vacuum step comprises less than 10 ppm nitrogen.

4. The process of claim 1 wherein reactor chamber effluent after the vacuum step comprises less than 1 ppm nitrogen.

5. The process of claim 1 wherein the sorption cycle operates at less than 50° C. and the conversion step operates at a temperature greater than 200° C.

6. The process of claim 1 wherein reactor temperature is raised from a vacuum step temperature to a beginning conversion step temperature, and, during said hydrogenation, the reactor temperature increases less than 20° C.

7. The process of claim 1 wherein reactor temperature is raised from a vacuum step temperature to a beginning conversion step temperature whereby said hydrogenation begins, and, as hydrogenation continues, the reactor temperature decreases.

8. The process of claim 1 wherein a preheater selected from a group consisting of an electric and a Joule heating exchanger is provided upstream of the reactor chamber to preheat a gas stream comprising hydrogen prior to flowing into the reactor chamber containing the catalyst and sorbent.

9. The process of claim 1 wherein said sub-atmospheric pressure is achieved using a vacuum pump.

10. The process of claim 1 wherein the catalyst and solid sorbent are coated on walls of open-ended flow channels of a catalyst substrate received in the reactor chamber.

11. The process of claim 10 wherein the catalyst comprises Ru.

12. The process of claim 10 wherein the sorbent comprises Na or Ca.

13. The process of claim 12 wherein the catalyst, the solid sorbent, and a porous support are a dual function material coating on the flow channels of the catalyst substrate and the sorbent is between 1 and 20 wt % of the coating.

14. The process of claim 1, wherein a reactor chamber effluent, during at least a portion of a single pass of said hydrogen being fed to the reactor chamber in the conversion step, is methane gas with a purity of greater than 90% on a dry basis.

15. The process of claim 1, wherein a reactor chamber effluent is recirculated to the reactor chamber during at least a portion of said conversion step and the process produces a reactor chamber product steam that is methane gas with a purity of greater than 90% on a dry basis.

16. A process for making methane from CO₂ and hydrogen, the process comprising providing a catalyst and sorbent provided in a reactor with a reactor length, the catalyst and sorbent being in contact for at least a portion of the reactor length, the process further comprising CO₂ being captured from a stream comprising the CO₂ and N₂ at an inlet N₂ concentration, and subsequently feeding a hydrogen stream to the reactor so that the captured CO₂ is hydrogenated to methane and a methane reactor effluent is produced, wherein the methane effluent contains an exit N₂ concentration less than 10 ppm N₂ and the exit N₂ concentration is lower than the inlet N₂ concentration.

17. The process of claim 16, wherein the methane reactor effluent contains less than 1% CO₂ and the reactor effluent is recirculated to the reactor during at least a portion of said feeding of a hydrogen stream to the reactor.

18. A method for making a combined sorbent and catalyst coating on interior surfaces of an open flow channel reactor body wherein the reactor body is made from an alumina forming material and the maximum flow channel opening is less than 2 mm in width or in height in at least a portion of the flow length.

19. The method of claim 18 wherein the reactor body is heat treated to at least 800° C. before coating the combined sorbent and catalyst onto said interior surfaces.

20. A process for making a hydrocarbon product from CO₂ and hydrogen using a reactor body comprising a sorbent and catalyst wherein the process comprises discontinuous flow of hydrogen to said reactor body, the flow of hydrogen occurring during a hydrogenation portion of the process wherein the temperature change within said reactor body is less than 20 C rise during said hydrogenation portion of the process.

21. The process of claim 20 where the hydrocarbon product is methane, so that said hydrogenation portion of the process comprises reaction of CO₂ and said hydrogen.

22. A method for producing high purity methane in a single cellular reactor from a gaseous stream comprising CO₂, the method comprising providing a cellular reactor having a length, and performing a step wherein the gaseous stream flows past a dual function material (DFM) material, on walls of a plurality of open-ended cells of the cellular reactor, along at least a portion of the reactor length, so that CO₂ is adsorbed, and performing a step comprising providing hydrogen in the reactor so that the adsorbed CO₂ and hydrogen are converted to methane and wherein effluent of the single cellular reactor is methane gas with a purity of greater than 90% on a dry basis upon a single pass of hydrogen.

23. The process of claim 22 where the effluent of the reactor is recirculated to the cellular reactor during at least a portion of said hydrogen provision to the reactor.

24. The method of claim 23 further comprising an additional step prior to said conversion to methane gas, the additional step comprising, after said CO₂ is sorbed, purging the reactor of non-sorbed species from the gaseous stream, the non-sorbed species comprising nitrogen.

25. The method of claim 24, wherein the method is a cyclic process wherein said gaseous stream comprising CO₂ flowing past the DFM so that CO₂ adsorbs, said purging, and said CO₂ and hydrogen converting to methane are repeated sequentially multiple times.

26. The method of claim 25, wherein purity of the produced methane is greater than 95%.

27. The method of claim 25, wherein purity of the produced methane is greater than 98%.

28. The method of claim 25, wherein concentration of nitrogen in the high purity methane is less than 1%.

29. The method of claim 25, wherein concentration of nitrogen in the high purity methane is less than 1%.

30. The method as in claim 25, wherein concentration of nitrogen in the high purity methane is less than 0.1%.

31. The method as in claim 25, wherein concentration of nitrogen in the high purity methane is less than 10 ppm.

32. The method as in claim 25, wherein concentration of nitrogen in the high purity methane is less than 2 ppm.

33. The method as in claim 25, wherein concentration of nitrogen in the high purity methane is less than 1 ppm.

34. A process for adjusting hydrogen content of a dual function material (DFM) effluent gas comprising hydrogen and gaseous hydrocarbons from a cyclic process producing the hydrocarbons over DFM, the process comprising using a hydrogen fuel cell on said DFM effluent gas and providing a fixed or variable electrical load, to alter the concentration of hydrogen in a fuel cell effluent gas, exiting the fuel cell, compared to said DFM effluent gas.

35. The process of claim 34 where the hydrocarbon is methane.

36. The process of claim 34 where hydrogen concentration in the fuel cell effluent gas is less than 10%.

37. The process of claim 34 where hydrogen concentration in the fuel cell effluent gas is less than 1%.

38. A process for using a reactor containing dual functional material, the reactor having two ends and a length between the two ends, the process comprising depositing current via at least one electrode to at least one of said two ends to allow said current to reach at least a portion of the reactor length where reactor feedstock flow traverses the length of the reactor, wherein at least one power source is provided outside the reactor and the at least one electrode is directly connected with the power source and spans at least a portion of the length of the reactor.
